(19) United States
(12) Patent Application Publication
(10) Pub. No.: US 2016/0319324 A1
(43) Pub. Date: Nov. 3, 2016

(54) MODULATORS OF GLYCEROPHOSPHODIESTER PHOSPHODIESTERASE PROTEINS

(71) Applicant: THE JOHNS HOPKINS UNIVERSITY, Baltimore, MD (US)

(72) Inventors: Shanthini Sockanathan, Baltimore, MD (US); Sungjin Park, Salt Lake City, UT (US)

(73) Assignee: THE JOHNS HOPKINS UNIVERSITY, Baltimore, MD (US)

(21) Appl. No.: 15/107,481

(22) PCT Filed: Dec. 23, 2014
(86) PCT No.: PCT/US2014/072030

§ 371 Date: Jun. 23, 2016

Related U.S. Application Data
(60) Provisional application No. 61/919,908, filed on Dec. 23, 2013.

(51) Int. Cl. C12Q 1/44; A61K 31/714; A61K 31/4402; A61K 31/165; A61K 31/192; A61K 31/7048; A61K 31/198

(52) U.S. Cl. CPC C12Q 1/44; C12Y 301/04046; A61K 31/7048; A61K 31/714; A61K 31/198; A61K 31/165; A61K 31/192; A61K 31/4402; G01N 2333/916; G01N 2500/20; G01N 2500/02

(57) ABSTRACT

The present invention relates to glycerophosphodiester phosphodiesterase (GDE) proteins. More specifically, the present invention relates to targeting GDE proteins to modulate its glycosylphosphatidylinositol (GPI)-cleaving activity. In a specific embodiment, the present invention provides a GDE modulator that modulates the surface GPI anchor cleavage activity of GDE.

### CROSS-REFERENCE TO RELATED APPLICATIONS

This application claims the benefit of U.S. Provisional Application No. 61/919,908, filed Dec. 23, 2013, which is incorporated herein by reference in its entirety.

### STATEMENT OF GOVERNMENTAL INTEREST

This invention was made with government support under grant no. NS046336 awarded by the National Institutes of Health. The government has certain rights in the invention.

### FIELD OF THE INVENTION

The present invention relates to glycerophosphodiester phosphodiesterase (GDE) proteins. More specifically, the present invention relates to targeting GDE proteins to modulate its glycosylphosphatidylinositol (GPI)-cleaving activity.

### INCORPORATION-BY-REFERENCE OF MATERIAL SUBMITTED ELECTRONICALLY

This application contains a sequence listing. It has been submitted electronically via EFS-Web as an ASCII text file entitled “P11856-04_ST25.txt.” The sequence listing is 815 bytes in size, and was created on Dec. 23, 2014. It is hereby incorporated by reference in its entirety.

### BACKGROUND OF THE INVENTION

During embryogenesis, the six-transmembrane protein GDE2 triggers postmitotic spinal motor neuron differentiation by utilizing extracellular glycerophosphodiester phosphodiesterase (GDPD) activity to downregulate Notch signaling in adjacent motor neuron progenitors. Conventional GDPDs hydrolyze glycerophosphodiesters into glycerol-3-phosphate and corresponding alcohols; however, whether GDE2 utilizes this mechanism to inhibit Notch signaling is unclear.

### SUMMARY OF THE INVENTION

Six-transmembrane glycerophosphodiester phosphodiesterase (GDE) proteins play key roles in multiple cellular processes within the organism including regulating cellular differentiation in the developing nervous system. Central to their function is their extracellular glycerophosphodiester phosphodiesterase (GDPD) activity. Several recent reports suggest that the six transmembrane GDEs utilize classical GDPD enzymatic activity for their function. The present invention is based, at least in part, on the discovery that the six transmembrane GDEs do not function as classical GDPDs but instead utilize a novel enzymatic mechanism to regulate key signaling pathways in the developing organism.

More specifically, the present inventors have discovered that GDE cleaves glycosylphosphatidylinositol (GPI) anchors. Although GPI-anchored proteins and their cleaved products have been implicated in various cellular processes and in disease conditions, the identity of a mammalian surface GPI-cleaving enzyme is not known to date. The present invention identifies for the first time the six-transmembrane GDPD proteins as the first surface GPI-cleaving enzymes in a mammalian system.

Thus, this first identification of a vertebrate specific GPI-cleaving family of enzymes provides a new family of targets for therapies involving diseases and cellular processes that involve GPI-anchored proteins. Identification of compounds that modulate GDE function could potentially directly increase or decrease the cleavage of GPI-anchored proteins thereby altering their function in diseases such as prion-related protein diseases of the brain and various cancers such as hepatocellular carcinoma (HCC). Also, GPI-anchored proteins function in various brain regions such as the hypothalamus and appetite centers where one could conceivably regulate appetite through administering compounds. GDE2 may be involved in synaptic plasticity, thus it is possible that compounds could modulate cognition, learning and memory. GDE3 may also be involved.

The present inventors have developed a high throughput screen to identify modulators of GDE activity and have identified preliminary agonists and antagonists of function

Accordingly, in one aspect, the present invention provides GDE modulators. In a particular embodiment, a GDE modulator is provided that modulates the GPI anchor cleavage activity of GDE. In certain embodiments, the modulator is a small molecule, an antibody, polypeptide, a polynucleotide, an aptamer, or an siRNA. In particular embodiments, the modulator is selected from the group consisting of erythromycin, vitamin B12, thyroxine, nonivamide, fenbufen and pyrilamine.

In another aspect, the present invention provides methods of screening for antagonists of GDE protein. In one embodiment, a method of screening for antagonists of a glycerophosphodiester phosphodiesterase (GDE) protein comprises the steps of (a) contacting a test agent with a cell that expresses the GDE protein; and (b) measuring the level of cleavage of glycosylphosphatidylinositol (GPI) anchors in the cell, wherein a test agent that decreases the measure cleavage level as compared to cleavage activity in a cell not contacted with the test agent identifies the test agent as an antagonist of GDE protein. In a specific embodiment, the GDE protein is GDE2 or GDE3. In other embodiments, the GDE protein is a GDE protein that cleaves GPI anchors. In certain embodiments, the test agent is a small molecule, an antibody, polypeptide, a polynucleotide, an aptamer, or an siRNA. In a particular embodiment, a method for treating a GDE-related disease, disorder or condition comprises the step of administering to a patient an effective amount of an antagonist identified herein. In certain embodiments, the antagonist is erythromycin, vitamin B12, thyroxine, or nonivamide.

In another aspect, the present invention provides methods of screening for agonists of GDE protein. In one embodiment, a method of screening for agonists of a GDE protein comprises the steps of (a) contacting a test agent with a cell that expresses the GDE protein; and (b) measuring the level of cleavage of GPI anchors in the cell, wherein a test agent that increases the measure cleavage level as compared to cleavage activity in a cell not contacted with the test agent identifies the test agent as an agonist of GDE protein. In a specific embodiment, the GDE protein is GDE2 or GDE3. In other embodiments, the GDE protein is a GDE protein that cleaves GPI anchors. In certain embodiments, the test agent is a small molecule, an antibody, polypeptide, a polynucleotide, an aptamer, or an siRNA. In a particular embodiment, a method for treating a GDE-related disease, disorder or condition comprises the step of administering to a patient an effective amount of an agonist identified herein. In particular embodiments, the agonist is fenbufen or pyrilamine.

In another embodiment, a method for identifying a GDE protein modulator comprising the step of measuring GPI anchor cleavage activity of the GDE protein in the presence and absence of a test agent, wherein an agent that increases or decreases cleavage activity relative to cleavage activity in the absence of the test agent identifies the test agent as a GDE protein modulator. In a specific embodiment, the GDE protein is GDE2 or GDE3. In other embodiments, the GDE protein is a GDE protein that cleaves GPI anchors. In certain embodiments, the test agent is a small molecule, an antibody, polypeptide, a polynucleotide, an aptamer, or an siRNA. In a particular embodiment, a method for treating a GDE-related disease, disorder or condition comprises the step of administering to a patient an effective amount of a modulator identified herein. In particular embodiments, the modulator is selected from the group consisting of erythromycin, vitamin B12, thyroxine, nonivamide, fenbufen, pyrilamine, and derivatives or biologically active fragments of the foregoing.

In a further embodiment, a method of screening for GDE modulators comprises the steps of (a) contacting a cell that expresses GDE with a test agent; (b) assaying GPI anchor cleavage by GDE; and (c) comparing the assayed GDE activity to GDE activity in a cell that has not been contacted with the test agent, wherein a difference in the compared GDE activity identifies the test agent as a GDE modulator. In a specific embodiment, the GDE protein is GDE2 or GDE3. In other embodiments, the GDE protein is a GDE protein that cleaves GPI anchors. In certain embodiments, the test agent is a small molecule, an antibody, polypeptide, a polynucleotide, an aptamer, or an siRNA. In a particular embodiment, a method for treating a GDE-related disease, disorder or condition comprises the step of administering to a patient an effective amount of a modulator identified herein. In particular embodiments, the modulator is selected from the group consisting of erythromycin, vitamin B12, thyroxine, nonivamide, fenbufen, pyrilamine, and derivatives or biologically active fragments of the foregoing.

In a specific embodiment, a method of screening for therapeutic agent useful in the treatment of GDE-mediated diseases comprises the steps of (a) contacting a test agent with a GDE polypeptide; and (b) detecting the binding of the test agent to the GDE polypeptide. In another embodiment, the method further comprises (c) contacting the test agent with a cell derived from a patient suffering from a GDE-mediated disease; and (d) determining the effect of the test agent on the cell. In a specific embodiment, the GDE polypeptide is GDE2 or GDE3. In other embodiments, the GDE polypeptide is a GDE polypeptide that cleaves GPI anchors. In certain embodiments, the test agent is a small molecule, an antibody, polypeptide, a polynucleotide, an aptamer, or an siRNA. In a particular embodiment, a method for treating a GDE-related disease, disorder or condition comprises the step of administering to a patient an effective amount of a therapeutic agent identified herein. In particular embodiments, the therapeutic agent is selected from the group consisting of erythromycin, vitamin B12, thyroxine, nonivamide, fenbufen, pyrilamine, and derivatives or biologically active fragments of the foregoing.

### BRIEF DESCRIPTION OF THE FIGURES

FIG. 1. GDE2 stimulates Dll1 shedding. (A-H) Confocal images of coronal section of E13.5 mouse spinal cords. Arrows in panels D, H mark red cells (Gata2/3+ Lhx3−) corresponding to V2b interneurons. (I) Graph quantifying the numbers of interneurons in WT and Gde2−/− mutants; mean±s.e.m, n=4, Student's t-test: V0 *p=0.0016; V1 p=0.4778; V2a *p=0.0028, V2b *p=0.0088; Schematic of the ventral spinal cord summarizing Gde2−/− phenotypes, VZ=ventricular zone; MZ=marginal zone; FP=floorplate; P=progenitor. (J) Western blot of Jag1 processing (FL=full length; CTF=C-terminal fragment) and quantification of Jag1 CTF/FL ratios from E12.5 embryonic spinal cord extracts. (K) Western blots of extracts of chick spinal cords electroporated with C-terminal Flag-tagged mouse Dll1 constructs; open arrow shows processed Dll1, black arrow marks Dll1-42. Arrow for GDE2 marks endogenous glycosylated GDE2, lower bands are hypoglycosylated versions of GDE2. (L) Western blots of electroporated spinal cord extracts (SC) and transfected HEK293T cells. Arrows are as in K. (M-O) Close up of VZ of chick spinal cords electroporated on the right side with Dll1, GDE2 or GDE2+Dll1. Arrow marks the midline.

FIG. 2. RECK ablation phenocopies GDE2 overexpression. (A-F) In situ hybridization of coronal sections of HH st19/20 chick spinal cords electroporated on the right with control (CshRNA) or shRNAs against RECK. * marks reduced expression. (G-L) Close up of VZ of chick spinal cords electroporated on the right side with control and RECK shRNAs. Arrow marks the midline; double headed arrow marks the extent of the VZ. (M) Western blots of electroporated chick spinal cords showing that ablating RECK stimulates Dll1-42 production (arrow) but does not affect Jag1 expression or processing. Graph quantifying Dll1-42 cleavage from Westerns; mean±s.e.m. Students t-test, n=4, sh1RECK *p=0.0066 sh2RECK *p=0.0175.

FIG. 3. GDE2 cleaves RECK within the GPI-anchor. (A) Schematic of conventional 2-step GDPD catalysis. (B) Graph quantifying in vitro GDPD assay in transfected HEK293T cells using glycerophosphoserine (GPserine) and synthetic cyclic G[1,2] phosphate intermediate as substrates. V=vector. (C-F) Western blots of transfected HEK293T cell lysates (lys) and media (med). (C) RECK is detected in the media in presence of catalytically active GDE2. (D) After sequential Triton X-114 extraction cleaved RECK from the media is consistently observed in the Detergent (DT)-free hydrophilic phase, while Dll1, which is not cleaved by GDE2, is retained in DT-rich hydrophobic phase of the lysate. (E) Schematic showing RECK induction (32 hours) followed by RECK silencing and GDE2 expression upon 4-OHT addition (16 hours). Western blots show that sequential expression of RECK and GDE2 led to RECK release in the media, whereas the same experiment with RECK and GDE1 did not. (F) RECK ECD generated by GDE2 expression or Pi-PLC incubation are similar. (G) Graphs quantifying amount of radiolabel incorporated into RECK or secreted (s) RECK in the presence of GDE2 or Pi-PLC. Mean±s.e.m. n=4-12. Schematic of GPI anchor. (H) Western blots of IPs of RECK from transfected HEK293T cells shows reactivity with CRD antibodies only after treatment with Pi-PLC.

FIG. 4. GDE2 inactivates RECK by GPI-cleavage. (A) Western blots of transfected HEK293T lysates (lys) and media (med). (B, C) Graphs quantifying ratio of ectopic Isl2+VZ motor neurons normalized to the number of transfected GDE2, or GDE2 and RECK. Representative images shown; arrows mark ectopic Isl2+ cells generated non cell-autonomously in the VZ by GDE2. All GDE2+ cells express RECK (data not shown). Mean+s.e.m., Students t-test, (B) *p=0.0306 compared with control (n=5); (C) *p=5.59×10⁻⁵ (n=8-10) compared with GDE2. In (B) suboptimal levels of RECK (RECKs/opt) were coelectroporated with GDE2 to assess strength of RECK suppression, in (C) high levels of RECK were coelectroporated with GDE2 to evaluate sufficiency of suppression. (D) Schematic of WT RECK with GPIanchor, RECK-CD2 (CD2 transmembrane domain replaces the GPI-anchor), sRECK (lacks GPI-anchor). (E) Western blot of extracts of chick spinal cords electroporated with shRNA1 directed against RECK 3′UTR to detect full-length (FL) and processed (Dll1-42) Dll1. The phenotype is rescued by exogenous plasmids expressing WT RECK ORF but not sRECK. Densitometric quantification of Dll1-42, mean±s.e.m., n=4. Students t-test, *p=0.013 compared with shRECK. (F) Model of GDE2 function. (FI) RECK in IZ cells prevents ADAM cleavage of Dll1 enabling Dll1/Notch binding, Notch activation and VZ progenitor maintenance. (FII) GDE2 in IZ cells cleaves RECK, enabling ADAM to cleave Dll1. Dll1-42 ECD is generated (2), active Dll1 is cleared from the membrane (1), which together downregulate Notch signaling in adjacent progenitors to initiate motor neuron differentiation.

FIG. 5. ADAM10 induces Dll1-42 in electroporated spinal cords. Western blots of protein extracts prepared from chick spinal cords electroporated with constructs expressing Dll1 and vector alone (empty), GDE2 or Adam10. Adam10 and Dll1 co-expression does not generate processed 30 kD Dll1 but a higher 50 kD form of Dll1. Nevertheless, Adam10 expression generates Dll1-42 as does GDE2 (arrow), suggesting that Adam activity lies downstream of GDE2.

FIG. 6. RECK expression and knockdown by shRNA. (A, B, D) Coronal sections of WT (A, B) and electroporated (D) chick spinal cords showing in situ hybridization of RECK transcripts. At HH st20 RECK mRNAs are detected in the ventricular zone (VZ) and in the intermediate zone where GDE2 resides; however, by HH st27, RECK expression is enriched in VZ cells. (C) Western blots of transfected HEK293T cells showing that shRNA1 directed against the chick RECK 3′UTR does not cause knockdown of RECK expressed from plasmids expressing the RECK ORF alone, whereas shRNA2 directed against the RECK ORF does; in both cases no knockdown of mouse RECK or GPI-anchored GFP is detected. No knockdown is observed on transfection of control shRNAs. (D) Electroporation of shRNA in chick spinal cords shows ablation of RECK expression. Right side is electroporated.

FIG. 7. RECK is localized to the cell surface after 32 hours. Western blot of total and surface biotinylated RECK 32 hours after transfection in HEK293T cells, prior to 4-OHT induced Cre-mediated excision of RECK and induction of GDE2.

FIG. 8. Six-transmembrane GDPD proteins release GPI-anchored proteins from the membrane. Western blots of protein extracts of transfected HEK293T cells showing that all members of the six transmembrane GDE family, i.e. GDE2, GDE3 and GDE6 can release the ECDs of GPI-anchored glypican 4 (GPC4) and GPI-anchored glypican 2 (GPC2) into the media.

FIG. 9. Evaluation of high throughput screens. GPI-anchored Luc-CNTFR was transfected to HEK293 cells with and without GDE3. Release index (luciferase activity of in the medium/cell lysate) were measured and the quality of the screens evaluated. Signal to noise ratio (S/N) is 44.6. Signal to background ratio (S/B) is 7.0. Z score is 0.6. n=6 samples. p=1.2E-09 by two-tailed student t test.

FIG. 10. Validation and identification of the site of action of candidate drugs. Drugs hit by a high-throughput screening were prepared from the fresh powders and their activity was tested by established western blot analysis. Erythromycin inhibits the release of CNTFR without affecting the lysate level, while mycophenolic acid and benzotropine affect glycosylation pattern of CNTFR. Concentration of drugs=10 μM.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The transition from cellular proliferation to differentiation is tightly controlled to ensure appropriate numbers of distinct cell types are formed, and to prevent the depletion or uncontrolled proliferation of progenitor cells. In spinal motor neuron development, GDE2 is necessary and sufficient to induce the generation of motor neuron subtypes through GDPD-dependent downregulation of Notch signaling in neighboring Olig2+ motor neuron progenitors (1-4). Normally, Notch is activated by the sequential processing of ligand-bound Notch receptors that release the Notch intracellular domain (NICD) to the nucleus to activate target gene expression (5). Since GDE2 functions non cell-autonomously to inhibit Notch signaling, the present inventors hypothesized that GDE2 does not directly regulate Notch processing but instead might affect the Notch ligands Delta (Dll) and Jagged (Jag) (3, 5).

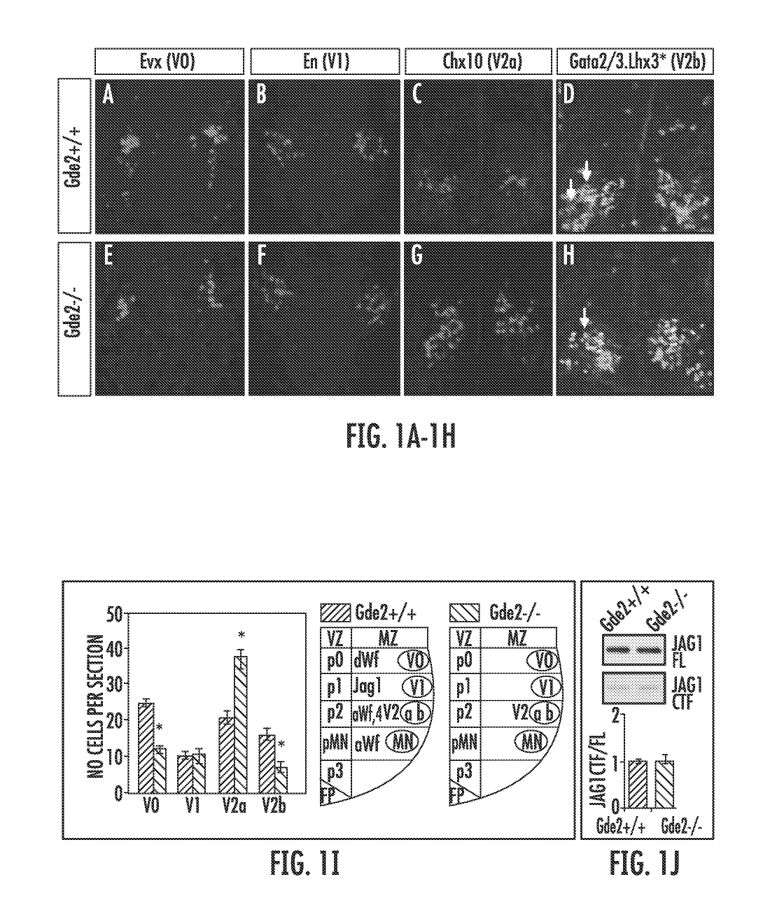

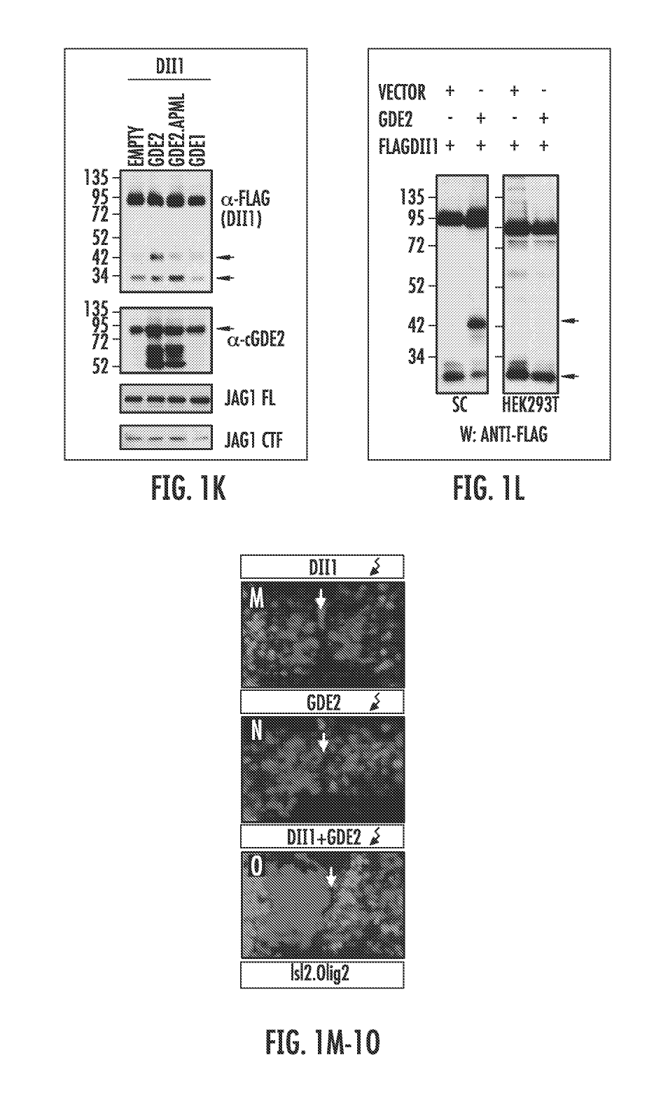

Jag1 and Dll1 are expressed in non-overlapping domains within the ventral spinal cord, and genetic ablation of either ligand causes domain-specific precocious neuronal differentiation (FIG. 1I) (5, 6). To explore the relationship between GDE2, Jag1 and Dll1, the present inventors determined the consequences of genetic ablation of GDE2 on spinal neuronal differentiation. Spinal cords of mice ablated for GDE2 (Gde2−/−) showed a specific loss of motor neurons and V0 interneurons, no changes in the total number of V1 interneurons or V2 interneurons, but an increase in the ratio of V2a:V2b interneurons (FIG. 1A-I; 3). Strikingly, these domain-specific deficits correlate with altered Dll1 but not Jag activity, suggesting that GDE2 specifically targets Dll1 function. While the motor neuron and V0 decrease in Gde2−/− animals are consistent with increased Notch signaling, the V2 interneuron phenotype resembles Notch inactivation (7); these collective observations imply that GDE2 can perturb Dll mediated Notch signaling according to cellular context.

Dll1 is inactivated through cleavage and release of its extracellular domain (ECD) by the ADAM family of metalloproteases (5). To determine if GDE2 GDPD activity promotes Dll1 shedding, the present inventors coelectroporated GDE2 and Dll1 into chick spinal cords and analyzed Dll1 processing by Western blot. Coexpression of GDE2 and Dll1 in chick spinal cords generated processed (30 kD) and full-length (95 kD) exogenous Dll1 (FIG. 1K). Moreover, GDE2/Dll1 coexpression induced an additional 42 kD Dll1 product (Dll1-42) that was not detected on coexpression of the two-pass transmembrane GDPD protein GDE1 (4, 8), or by catalytically inactive GDE2 GDPD mutants (GDE2.APML; 3) (FIG. 1K). Consistent with the Dll1-specific function of GDE2, no changes in Jag1 expression (JagFL) or processing (JagCTF) were detected in electroporated spinal cords or in spinal cords of Gde2−/− animals (FIG. 1J, K). These data suggest that GDE2 GDPD activity stimulates Dll1 processing to form Dll1-42. GDE2 did not induce Dll1-42 when coexpressed with Dll1 in heterologous HEK293T cells, suggesting that GDE2-dependent processing of Dll1 is indirect (FIG. 1L). In support of this finding, ADAM10 and Dll1 coexpression in chick spinal cords robustly induced Dll1-42, suggesting that Dll1-42 is generated through ADAM metalloprotease activity (FIG. 5).

Dll1 processing inhibits Notch signaling by removing available membrane-bound Dll1 for Notch receptor binding and also by a dominant-negative activity associated with the released Dll1 ECD (5); thus, Dll1 should function cooperatively with GDE2 to induce motor neuron differentiation. Indeed, coexpression of GDE2 and Dll1 in chick spinal cords significantly enhanced the ability of GDE2 to induce the premature differentiation of VZ progenitors into Isl2+ motor neurons (FIG. 1M-O). Taken together, these observations support the model that GDE2 GDPD activity promotes motor neuron differentiation by stimulating ADAM-dependent Dll1 processing.

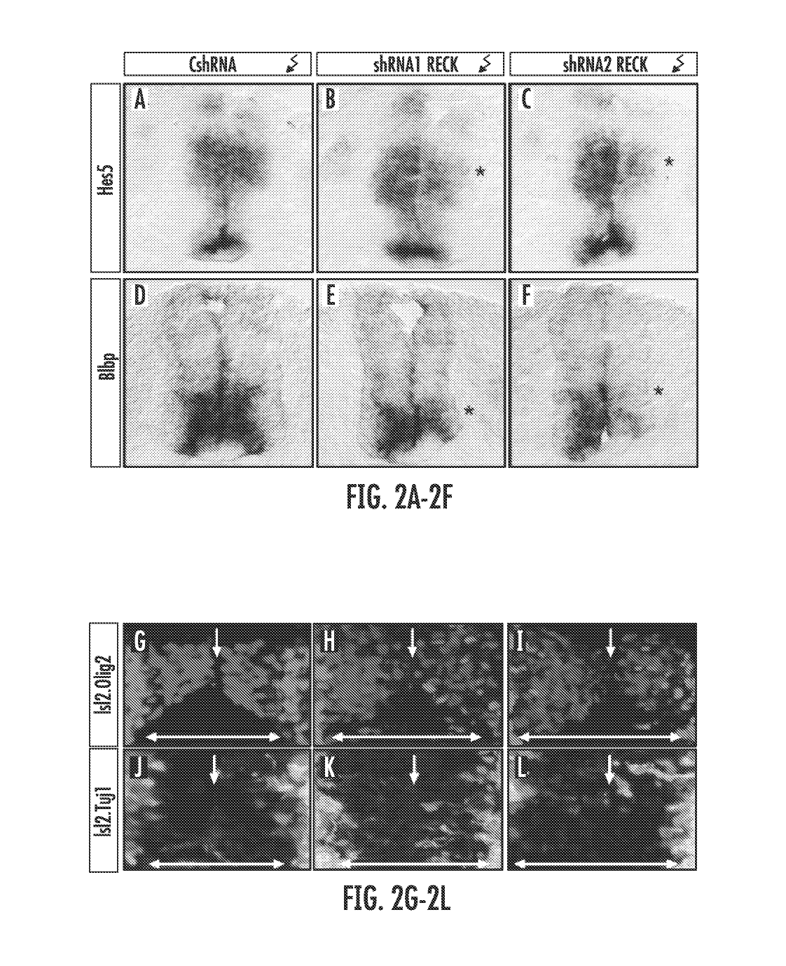

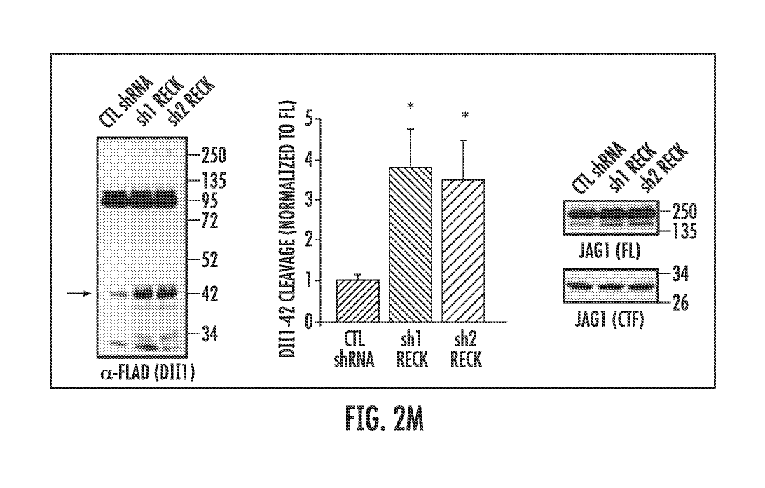

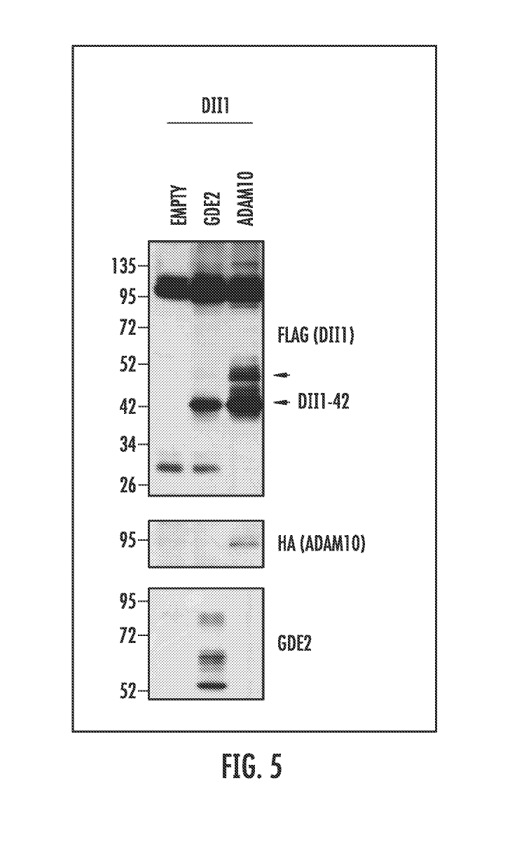

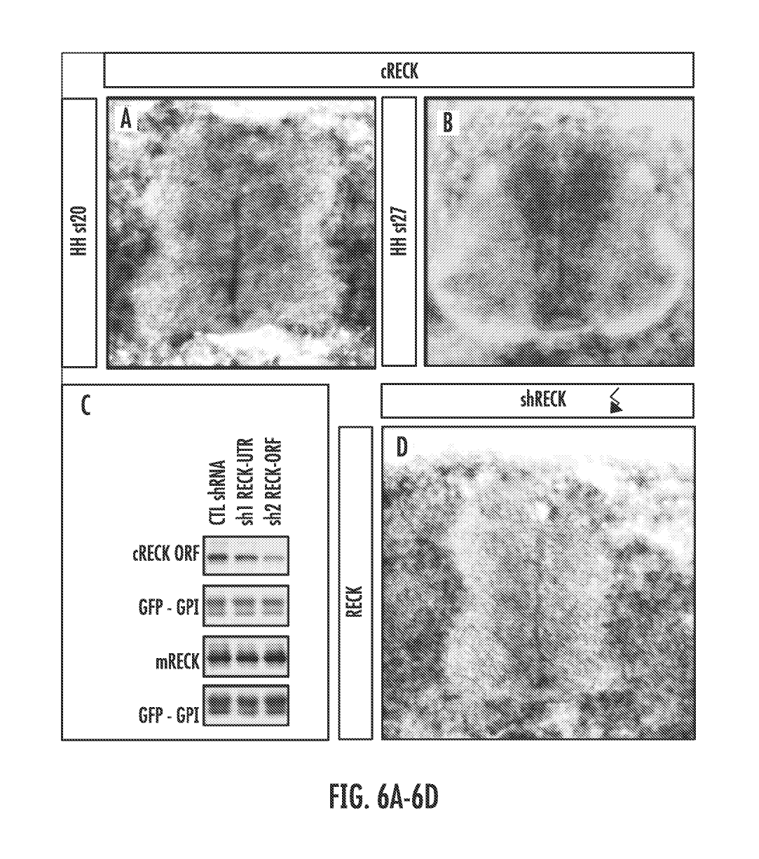

The present inventors noted that the GPI-anchored protein RECK, which is implicated in multiple cancers, activates Notch signaling in cortical progenitors by directly inhibiting the proteolytic activity of ADAM10 on Dll1 (9, 10). To investigate the possibility that RECK is a target of GDE2 GDPD activity, the present inventors examined the distribution of RECK transcripts in the developing spinal cord. RECK mRNA is enriched and maintained in VZ and IZ cells throughout the period of motor neuron generation, and thus overlaps with GDE2 expression in newly differentiating motor neurons (FIG. 6A, B). RECK ablation in spinal cords by two different shRNAs (FIG. 6C, D) significantly lowered Notch signaling as assayed by reduced expression of the Notch target genes Hes5 and Blbp (5) and the induction of premature motor neuron differentiation in the VZ (FIG. 2A-L). Moreover, loss of RECK specifically induced Dll1-42 in spinal cords but did not alter Jag1 expression and processing to Jag1 CTF (FIG. 2M). These phenotypes are strikingly similar to when GDE2 is overexpressed, and suggest that GDE2 GDPD activity promotes Dll1 shedding by inactivating RECK (1-3).

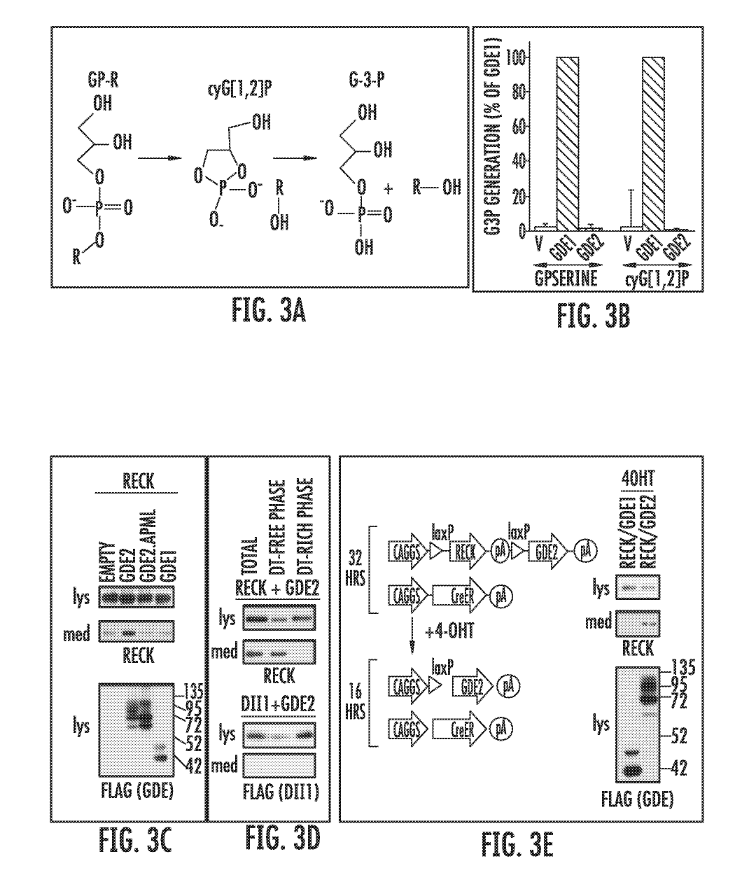

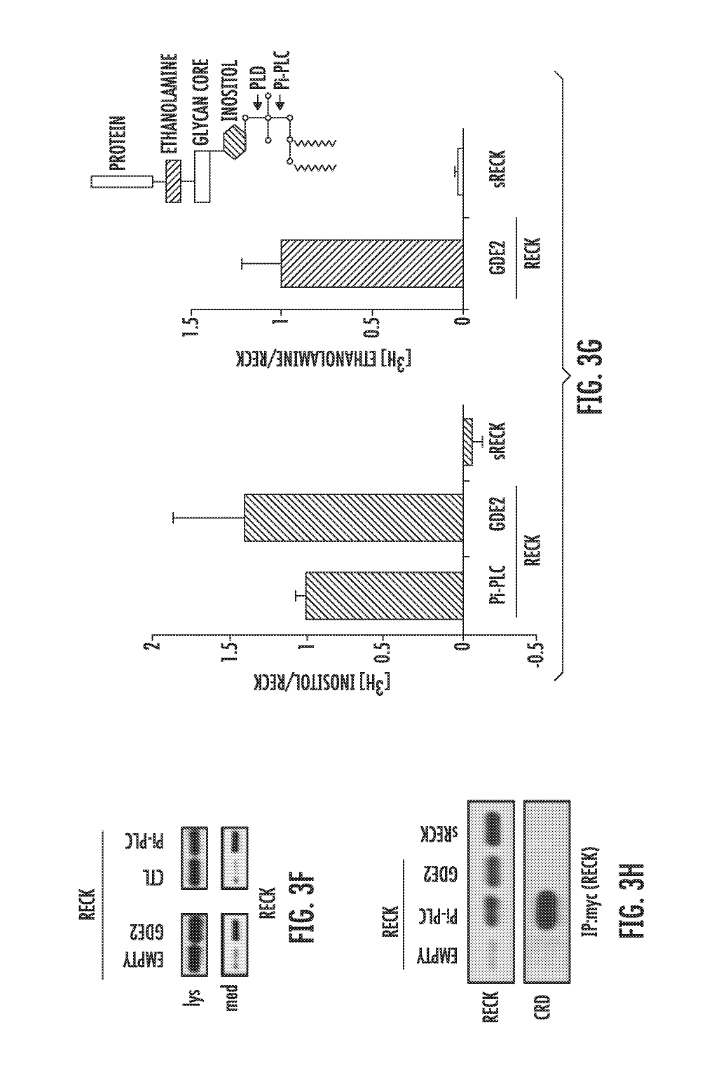

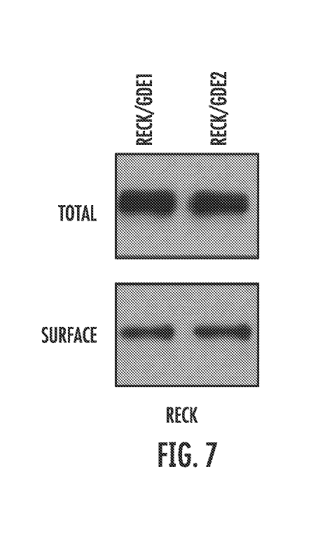

To define the mechanism of GDE2 function and investigate how it might inactivate RECK, the present inventors examined if GDE2 exhibits conventional GDPD phospholipase-D (PLD) catalysis using a well-characterized coupled spectrophotometric assay of GDPD function (FIG. 3A, B) (11, 12). Membrane fractions prepared from HEK293T cells transfected with control GDE1 showed robust GDPD activity when incubated with glycerophosphoserine (GP-S), or a cyclic glycerol-1,2-phosphate intermediate (cyG[1,2]P) that is not substrate-specific and formed by GDPD enzymes during their predicted 2-step catalytic mechanism (FIG. 3A, B; 11-13). In contrast, GDE2 showed no detectable GDPD activity in both cases, suggesting that GDE2 does not exhibit conventional GDPD PLD activity under these conditions (FIG. 3B). The GDPD domains of the six-transmembrane GDEs (GDE2, GDE3 and GDE6) are homologous to the catalytic X-domain of Pi-PLC, and GDE3 hydrolyzes glycerophosphoinositol via a PLCtype cleavage mechanism (14, 15). Given that Pi-PLCs cleave and release GPI-linked proteins from membranes, the present inventors tested the possibility that GDE2 GDPD activity inactivates RECK by GPI-cleavage. The present inventors co-expressed GDE2 and RECK in HEK293T cells and assayed the media for cleaved RECK ECD by Western blots. GDE2 and RECK coexpression led to the release of RECK into the media, while media prepared from cells transfected with vector alone, GDE1 or GDE2.APML contained little to no RECK (FIG. 3C). Repeated Triton X-114 extraction of the media from cells co-expressing GDE2 and RECK reproducibly detected RECK in hydrophilic fractions, ruling out potential media contamination by membrane-bound RECK (FIG. 3D; 16). No differences in cell viability between cells co-transfected with RECK and GDE2, GDE2.APML or GDE1 were found, suggesting that RECK detected in the media is not due to cell lysis (data not shown). Further, sequential induction and inhibition of RECK expression to generate surface-bound RECK (FIG. 7), followed by GDE2 expression showed similar release of RECK into the media (FIG. 3E). These observations suggest that GDE2 acts on surface GPI-anchored RECK and does not promote aberrant RECK discharge through disruption of RECK synthesis, modification or transport.

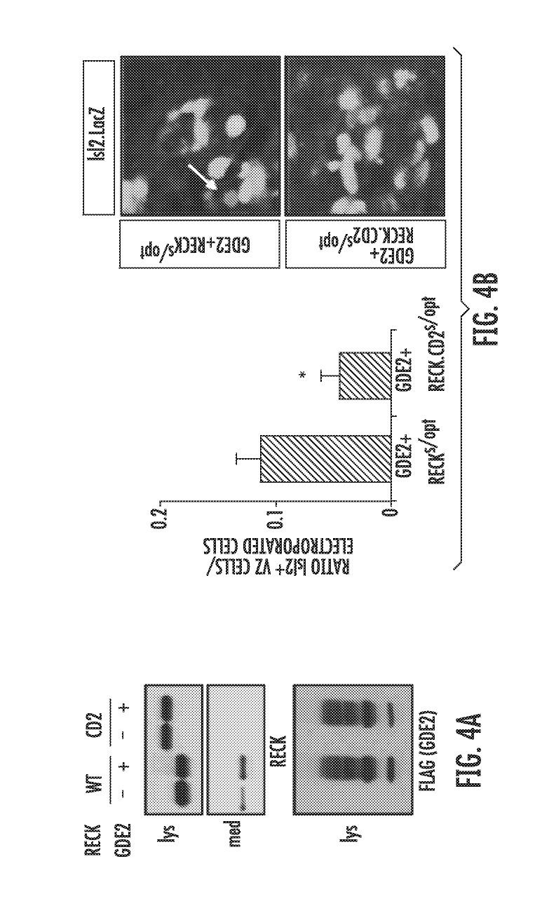

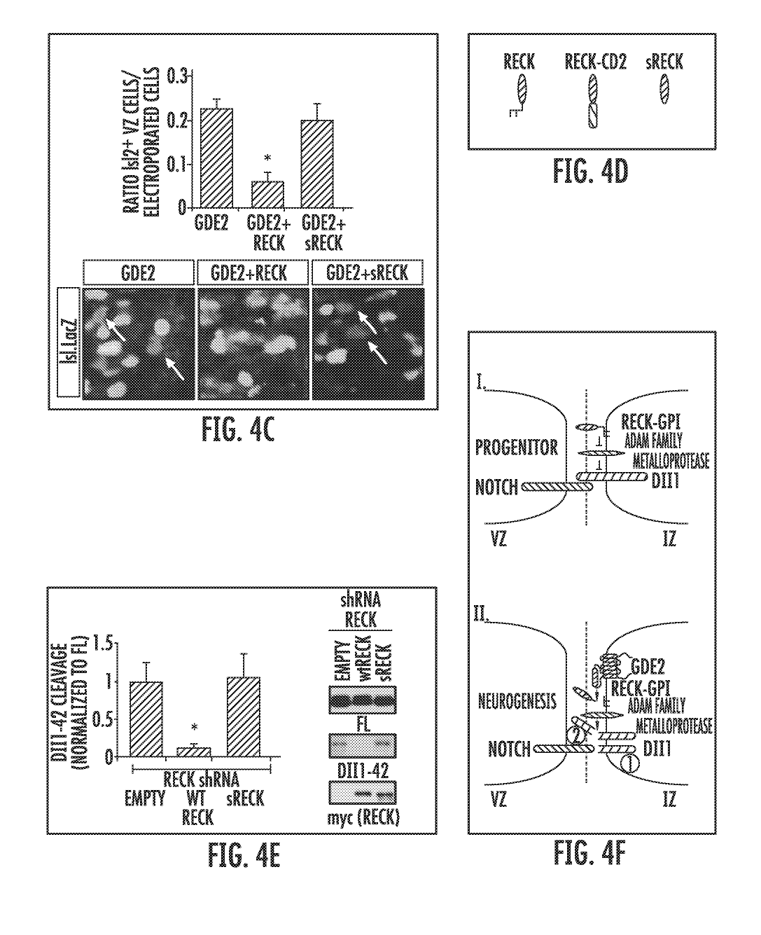

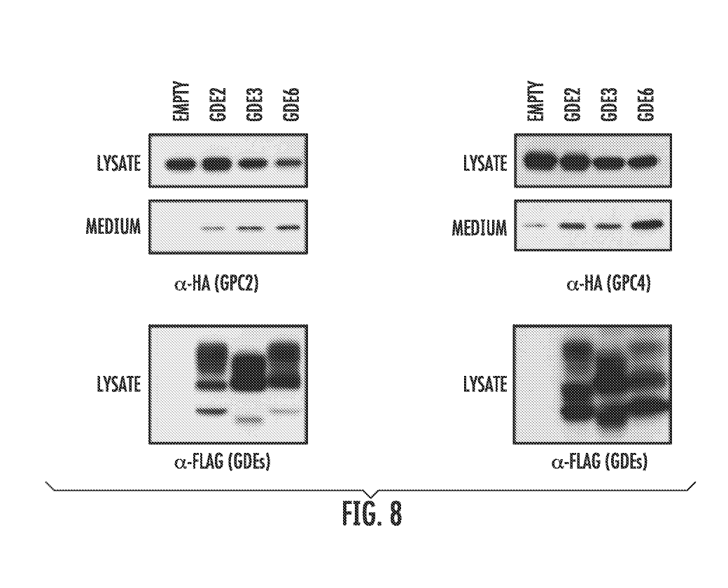

RECK ECD released by GDE2 GDPD activity co-migrated with RECK generated by Pi-PLC treatment of RECK-transfected cells (FIG. 3F). Pi-PLC cleavage of GPI linkages reveals a 1,2 cyclic inositol phosphate ring (cyIno[1,2]P) that is the major epitope recognized by CRD antibodies and is widely used to identify classical Pi-PLC cleavage (17, 18). Strikingly, RECK ECD arising from GDE2 co-expression showed no reactivity to CRD antibodies in contrast to RECK ECD generated from Pi-PLC treatment (FIG. 3H). However, whether GDE2 GDPD activity cleaves the GPI-linkage via a PLC type mechanism remains an open question as GDPD hydrolysis of cyIno[1,2]P could occur too rapidly to detect by this method (FIG. 3A). If GDE2 GDPD activity cleaves RECK within the GPI-anchor, then RECK ECD generated by GDE2 should contain ethanolamine and inositol residues (FIG. 3G). Radiolabeling of transfected HEK293T cells showed that RECK ECD released into the media by Pi-PLC treatment or by GDE2 expression contained equivalent levels of [3H] inositol, whereas a secreted version of RECK ECD (sRECK) that lacks the GPI anchor was poorly labeled under identical conditions (FIG. 3G, FIG. 4D; 19). Similar results were obtained when cells were incubated with [3H] ethanolamine (FIG. 3; 19). Substitution of the RECK GPI-anchor with the membrane tethering domain from the non-GPI-anchored CD2 protein failed to produce RECK in the media in the presence of GDE2 (FIG. 4A, D; 20, 21). Taken together, these observations suggest that GDE2 GDPD activity cleaves within the GPI-anchor to release RECK from the membrane; this mechanism is further supported by the ability of GDE2 to cleave other unrelated GPI-anchored proteins such as GPC2 and GPC4 (FIG. 8).

If GDE2 inactivates RECK to induce motor neuron differentiation, then overexpression of RECK should overcome GDE2 inhibition and suppress GDE2− dependent induction of premature motor neuron generation in the VZ. Established Crelox techniques to induce sparse expression of GDE2 in Olig2+ motor neuron progenitors, elicits neighboring cells to differentiate into Isl2+ motor neurons (3, 22). Titration of WT GPI-anchored RECK coexpressed with GDE2 effectively suppressed GDE2-dependent premature motor neuron differentiation (FIG. 4C). Strikingly, RECK-CD2 exhibited a stronger suppression of GDE2 induction of motor neuron differentiation when compared to equivalent levels of GPI-anchored RECK (FIG. 4B). These data provide further evidence that GDE2 inactivates RECK to induce motor neuron differentiation through cleavage of the GPI-anchor.

One prediction of the present inventors' model is that the RECK ECD generated after GPI-cleavage should be inactive and thus incapable of maintaining Olig2+ motor neuron progenitors via Notch activation. However, soluble versions of RECK that lack the GPI-anchor are active in other systems, raising the possibility that the activity of cleaved RECK is context dependent (9). To test RECK ECD activity in terms of motor neuron differentiation, the present inventors compared the ability of WT RECK and sRECK to inhibit GDE2-dependent motor neuron generation in electroporated chick spinal cords (FIG. 4C, D). As described above, WT RECK coexpressed with GDE2 effectively suppressed GDE2-dependent premature motor neuron differentiation (FIG. 4C); in contrast, sRECK failed to suppress GDE2 activity (FIG. 4C). Further, analyses of electroporated chick spinal cords showed that WT RECK was sufficient to prevent increased Dll1 shedding resulting from ablation of endogenous RECK by shRNAs, whereas sRECK had no effect (FIG. 4E). These observations suggest that RECK ECD is non-functional in terms of inhibiting Dll1 shedding and imply that GDE2 GDPD-dependent cleavage of RECK clears active RECK from the membrane.

The present data suggest a model where GDE2 promotes motor neuron differentiation by inactivating surface bound RECK through GPI-cleavage, and accordingly, derepressing ADAM protein function. ADAM metalloprotease activity subsequently stimulates Dll1 shedding and downregulation of Notch signaling in adjacent motor neuron progenitors, thus triggering their differentiation into postmitotic motor neurons (FIG. 4F). This study shows that in the context of motor neuron differentiation, GDE2 GDPD function downregulates Notch signaling; however, our analysis of Gde2 null animals suggests that GDE2 can also activate Notch signaling to diversify V2 interneurons, conceivably through cleavage of other GPI-anchored proteins.

Three mammalian GPI-cleaving enzymes have been identified to date but none of them are membrane-bound; moreover, only the secreted protein Notum cleaves GPI-anchored proteins at the cell surface, as GPI-PLD fails to do so and Angiotensin-Converting Enzyme (ACE) GPI-cleaving activity remains controversial (23-25). Strikingly, the present inventors find that GDE2 GDPD activity cleaves various unrelated GPI-proteins as does its family members, GDE3 and GDE6 (FIG. 8; 4). Although further analysis is warranted, the present inventors' observations raise the intriguing possibility that the six-transmembrane GDPDs constitute a novel vertebrate-specific family of membrane-bound GPI-cleaving proteins that directly modulates GPI-anchored protein function. GPI-anchored proteins are key regulators of major signaling pathways such as Notch, FGFs, Wnts and Shh that play central roles in controlling diverse biological processes throughout the developing and adult organism (26, 27). Deeper understanding of how these pathways are regulated through GPI-cleavage in normal and diseased states will be gained by further analysis of six-transmembrane GDE GDPD protein expression, transport and activity.

### I. DEFINITIONS

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “GDE modulator” refers to an agent that modulates a GDE protein family member whose activity comprises cleavage of GPI anchors and specifically includes GDE. It is understood that reference to a “GDE modulator” also means reference to modulators of GDE proteins that cleave GPI anchors. GDE modulators include modulators that modulate GDE2, GDE3, etc., collectively or individually. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is useful in the treatment of a disease disclosed herein.

As used herein, an “antagonist” is a type of modulator and the term refers to an agent that binds a target (e.g., a protein) and can inhibit one or more functions of the target. For example, an antagonist of a protein can bind the protein and inhibit the binding of a natural or cognate ligand to the protein and/or inhibit signal transduction mediated through the protein.

An “agonist” is a type of modulator and refers to an agent that binds a target and can activate one or more functions of the target. For example, an agonist of a protein can bind the protein and activate the protein in the absence of its natural or cognate ligand.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against GDE and used as GDE modulators. Antibodies can be raised against other GDE family members that cleave GPI anchors.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Optional” or “optionally” means that the subsequently described event or circumstance can or cannot occur, and that the description includes instances where the event or circumstance occurs and instances where it does not.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with a GDE mediated disease, disorder or condition.

The terms “subject” and “patient” are used interchangeably here, and are intended to include organisms, e.g., eukaryotes, which are suffering from or afflicted with a disease, disorder or condition associated with GDE including, but not limited to GDE2 and GDE3. Examples of subjects or patients include mammals, e.g., humans, dogs, cows, horses, pigs, sheep, goats, cats, mice, rabbits, rats, and transgenic non-human animals. In certain embodiments, the subject or patient is a human, e.g., a human suffering from, at risk of suffering from, or potentially capable of suffering from, prion-related protein diseases of the brain and various cancers such as HCC, and other diseases or conditions described herein (e.g., a GDE-related disease, disorder or condition).

As used herein, the term “effective,” means adequate to accomplish a desired, expected, or intended result. More particularly, a “therapeutically effective amount” as provided herein refers to an amount of a GDE modulator of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a GDE modulator, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The terms “GDE-related disease, disorder or condition” or “GDE-mediated disease, disorder or condition,” and the like mean diseases, disorders or conditions associated with aberrant GDE signaling, including but not limited to cancers; other, non-oncogenic proliferative diseases, such as prion-related diseases of the brain. In certain embodiments, a GDE-related condition includes those conditions affecting cognition, learning and memory. In other embodiments, a GDE-related condition includes regulating appetite. GDE-related diseases, disorders or conditions include any abnormal state that involves GDE activity. The abnormal state can be induced by environmental exposure or drug administration. Alternatively, the disease or disorder can be due to a genetic defect. It is understood that reference to a “GDE-related disease, disorder or condition” also means reference to diseases, disorders or conditions related to GDE proteins that cleave GPI anchors. GDE can include, but it not limited to, GDE2 and GDE3.

### II. GDE POLYNUCLEOTIDES, POLYPEPTIDES AND EXPRESSION THEREOF

In particular embodiments, GDE is human. In other embodiments, GDE is non-human (e.g., primate, rodent, canine, or feline). There are a variety of sequences that are disclosed on GenBank, at www.pubmed.gov, and these sequences and others herein are incorporated by reference in their entireties as are individual subsequences or fragments contained therein. The nucleotide and amino acid sequences of the human GDE (including, but not limited to, GDE2 and GDE3) are publicly available. Thus provided are the nucleotide sequences of GDE as well as nucleotide sequences at least about 70%, 75%, 80%, 85%, 90%, 95%, 98%, 99% or more, identical to the publicly available nucleotide sequence. Also provided are amino acid sequences of GDE as well as amino acid sequence at least about 70%, 75%, 80%, 85%, 90%, 95%, 98%, 99% or more, identical to the publicly available nucleotide sequence.

As with all peptides, polypeptides, and proteins, including fragments thereof, it is understood that additional modifications in the amino acid sequence of the GDE polypeptides can occur that do not alter the nature or function of the peptides, polypeptides, or proteins. Such modifications include conservative amino acids substitutions.

The polypeptides described herein can be further modified and varied so long as the desired function is maintained. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99 percent identity to GDE and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989), Jaeger et al., Proc. Natl. Acad. Sci. USA 86:7706-7710 (1989), Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism) or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Post-translational modifications can include variations in the type or amount of carbohydrate moieties of the protein core or any fragment or derivative thereof. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from two to six residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place.

Modifications, including the specific amino acid substitutions, are made by known methods. By way of example, modifications are made by site-specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Nucleic acids that encode the polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

Isolated nucleic acid molecules can be produced by standard techniques. For example, polymerase chain reaction (PCR) techniques can be used to obtain an isolated nucleic acid containing a nucleotide sequence described herein. Various PCR methods are described, for example, in PCR Primer: A Laboratory Manual, Dieffenbach and Dveksler, eds., Cold Spring Harbor Laboratory Press, 1995. Various PCR strategies also are available by which the site-specific nucleotide sequence modifications described herein can be introduced into a template nucleic acid. Optionally, isolated nucleic acids are chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids disclosed herein also can be obtained by mutagenesis of, e.g., a naturally occurring DNA.

Nucleic acids that encode the polypeptide sequences, variants, and fragments thereof can be cloned into a vector for delivery into the cell. There are a number of compositions and methods which can be used to deliver nucleic acids to cells, either in vitro or in vivo via, for example, expression vectors. These methods and compositions can largely be broken down into two classes: viral based delivery systems and non-viral based delivery systems. Such methods are well known and readily adaptable for use with the compositions and methods described herein.

As used herein, plasmid or viral vectors transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, Sindbis and other RNA viruses, including these viruses with the HIV backbone. Also preferred are any viral families which share the properties of these viruses which make them suitable for use as vectors. Retroviral vectors, in general, are described by Verma, I. M., Retroviral vectors for gene transfer, in Microbiology-1985, American Society for Microbiology, Washington, pp. 229-232 (1985), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma and a number of other tissue sites. Other useful systems include, for example, replicating and host-restricted non-replicating vaccinia virus vectors.

Also provided are expression vectors comprising the disclosed nucleic acids, wherein the nucleic acids are operably linked to an expression control sequence. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Particular promoters controlling transcription from vectors in mammalian host cells may be obtained from various sources, for example, the genomes of viruses such as polyoma, Simian Virus 40 (SV40), adenovirus, retroviruses, hepatitis B virus and most preferably cytomegalovirus, or from heterologous mammalian promoters, e.g., beta actin promoter or EF1 promoter, or from hybrid or chimeric promoters (e.g., cytomegalovirus promoter fused to the beta actin promoter). The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs in length, and they function in cis. Enhancers usually function to increase transcription from nearby promoters. Enhancers can also contain response elements that mediate the regulation of transcription. While many enhancer sequences are now known from mammalian genes (globin, elastase, albumin, fetoprotein and insulin), typically one will use an enhancer from a eukaryotic cell virus for general expression. Examples of enhancers include the SV40 enhancer on the late side of the replication origin, the cytomegalovirus early promoter enhancer, the polyoma enhancer on the late side of the replication origin, and adenovirus enhancers.

Optionally, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A particular promoter of this type is the CMV promoter. Other promoters include SV40 promoters, cytomegalovirus (plus a linked intron sequence), beta-actin, elongation factor-1 (EF-1) and retroviral vector LTR. Optionally the promoter and/or enhancer region can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light.

The vectors also can include, for example, origins of replication, scaffold attachment regions (SARs), and/or markers. A marker gene can confer a selectable phenotype, e.g., antibiotic resistance, on a cell. This marker product is used to determine if the gene has been delivered to the cell and once delivered is being expressed. Examples of marker genes include the E. coli lacZ gene, which encodes B galactosidase, green fluorescent protein (GFP), and luciferase. Examples of suitable selectable markers for mammalian cells are dihydrofolate reductase (DHFR), thymidine kinase, neomycin, neomycin analog G418, hygromycin, blasticidin, and puromycin. When such selectable markers are successfully transferred into a mammalian host cell, the transformed mammalian host cell can survive if placed under selective pressure.

In certain embodiments, GDE (e.g., GDE2 or GDE3) is linked to an expression tag. An expression vector can include a tag sequence designed to facilitate manipulation or detection (e.g., purification or localization) of the expressed polypeptide. Tag sequences, such as glutathione S-transferase (GST), polyhistidine (His), myc, hemagglutinin (HA), V5, IgG, T7, or FLAG™ tag (Kodak, New Haven, Conn.) sequences typically are expressed as a fusion with the encoded polypeptide. For example, GDE can be linked to the IgG tag. Such tags can be inserted anywhere within the polypeptide, including at either the carboxyl or amino terminus. Optionally the expression tag can be a fluorescent protein tag. Fluorescent proteins can, for example, include such proteins as green fluorescent protein (GFP), red fluorescent protein (RFP), cyan fluorescent protein (CFP), yellow fluorescent protein (YFP), and blue fluorescent protein (BFP). Fluorescent proteins can be inserted anywhere within the polypeptide, but are most preferably inserted at either the carboxyl or amino terminus.

### III. GDE MODULATORS

In certain embodiments, the GDE modulator is selected from the group consisting of a small molecule, a polypeptide, a nucleic acid molecule, a peptidomimetic, or a combination thereof. In a specific embodiment, the agent can be a polypeptide. The polypeptide can, for example, comprise an extracellular domain of GDE2 (or of other GDE protein family members that cleave GPI anchors including GDE3). The polypeptide can also comprise an antibody. In another embodiment, the agent can be a nucleic acid molecule. The nucleic acid molecule can, for example, be a GDE inhibitory nucleic acid molecule. The GDE inhibitory nucleic acid molecule can comprise a short interfering RNA (siRNA) molecule (including, for example, a short hairpin RNA (shRNA)), a microRNA (miRNA) molecule, or an antisense molecule.

As used herein, a GDE inhibitory nucleic acid sequence can be a siRNA sequence or a miRNA sequence. A 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). An siRNA sequence preferably binds a unique sequence within the GDE mRNA with exact complementarity and results in the degradation of the GDE mRNA molecule. An siRNA sequence can bind anywhere within the mRNA molecule. An miRNA sequence preferably binds a unique sequence within the GDE mRNA with exact or less than exact complementarity and results in the translational repression of the GDE mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

As used herein, a GDE inhibitory nucleic acid sequence can be an antisense nucleic acid sequence. Antisense nucleic acid sequences can, for example, be transcribed from an expression vector to produce an RNA which is complementary to at least a unique portion of the GDE mRNA and/or the endogenous gene which encodes GDE. Hybridization of an antisense nucleic acid molecule under specific cellular conditions results in inhibition of GDE protein expression by inhibiting transcription and/or translation.

A GDE modulator can also be an antibody. The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

In other embodiments, a GDE (e.g., GDE2, GDE3, and the like) modulator is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Compound libraries may be screened for GDE modulators. A compound library is a mixture or collection of one or more putative modulators generated or obtained in any manner. Any type of molecule that is capable of interacting, binding or has affinity for GDE may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative modulator or member, i.e., a plurality of members or putative modulators. In certain embodiments, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular embodiments, a compound library may comprise less than about 50,000, 25,000, 20,000, 15,000, 10,000, 5,000, 1000, or 500 putative modulators.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.

The agents which are utilized in accordance with the method of the present invention may take any suitable form. For example, proteinaceous test agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both proteinaceous and non-proteinaceous test agents herein described may be linked, bound otherwise associated with any other proteinaceous or non-proteinaceous molecules. For example, in one embodiment of the present invention, the agent is associated with a molecule which permits its targeting to a localized region.

The test agent molecules may act either directly or indirectly to modulate the expression of GDE or the activity of the GDE expression product. The molecule acts directly if it associates with the GDE nucleic acid molecule or expression product to modulate expression or activity, respectively. The molecule acts indirectly if it associates with a molecule other than the GDE nucleic acid molecule or expression product which other molecule either directly or indirectly modulates the expression or activity of the GDE nucleic acid molecule or expression product, respectively. Accordingly, the method of the present invention encompasses the regulation of GDE nucleic acid molecule expression or expression product activity via the induction of a cascade of regulatory steps.

The term “expression” refers, for example, to the transcription and translation of a nucleic acid molecule. Reference to “expression product” is a reference to the product produced from the transcription and translation of a nucleic acid molecule.

“Derivatives” of the molecules herein described (for example GDE or other test agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, GDE or derivative thereof may be fused to a molecule to facilitate its entry into a cell. Analogues of the molecules contemplated herein include, for example, modification to side chains, incorporating of unnatural amino acids and/or their derivatives during peptide, polypeptide or protein synthesis and the use of crosslinkers and other methods including conformational constraints on the proteinaceous molecules or their analogues.

Derivatives of nucleic acid sequences which may be utilized in accordance with the method described herein may similarly be derived from single or multiple nucleotide substitutions, deletions and/or additions including fusion with other nucleic acid molecules. The derivatives of the nucleic acid molecules utilized as described herein include, for example, oligonucleotides, PCR primers, antisense molecules, molecules suitable for use in co-suppression and fusion of nucleic acid molecules. Derivatives of nucleic acid sequences also include degenerate variants.

A “variant” of GDE should be understood to include, for example, molecules that exhibit at least some of the functional activity of the form of GDE of which it is a variant. A variation may take any form and may be naturally or non-naturally occurring. A mutant molecule is one which exhibits, for example, modified functional activity.

A “homologue” is includes, for example, that the molecule is derived from a species other than that which is being treated in accordance with the method of the present invention. This may occur, for example, where it is determined that a species other than that which is being treated produces a form of GDE which exhibits similar and suitable differentiation or GPI anchor cleavage activity to that of the GDE which is naturally produced by the subject undergoing treatment.

Chemical and functional equivalents include, for example, molecules exhibiting any one or more of the functional activities of the subject molecule, which functional equivalents may be derived from any source such as being chemically synthesized or identified via screening processes such as natural product screening. For example chemical or functional equivalents can be designed and/or identified utilizing well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (e.g., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

In another embodiment, the test compounds is one or more of a peptide, a small molecule, an antibody or fragment thereof, and nucleic acid or a library thereof.

Also useful in the screening techniques described herein are combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing GDE analogues which exhibit properties such as more potent pharmacological effects.

With respect to high throughput library screening methods, oligomeric or small-molecule library compounds capable of interacting specifically with a selected biological agent, such as a biomolecule, a macromolecule complex, or cell, are screened utilizing a combinational library device which is easily chosen by the person of skill in the art from the range of well-known methods, such as those described above. In such a method, each member of the library is screened for its ability to interact specifically with the selected agent. In practicing the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of GDE, it may also be desirable to identify and utilize molecules which function agonistically or antagonistically to GDE in order to up or down-regulate the functional activity of GDE in relation to modulating GPI anchor cleavage. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of GDE capable of acting as agonists or antagonists. Chemical agonists may not necessarily be derived from GDE but may share certain conformational similarities. Alternatively, chemical agonists may be specifically designed to mimic certain physiochemical properties of GDE. Antagonists may be any compound capable of blocking, inhibiting or otherwise preventing GDE from carrying out its normal biological functions. Antagonists include monoclonal antibodies specific for GDE or parts of GDE.

Analogues of GDE or of GDE agonistic or antagonistic agents contemplated herein include, for example, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH.sub.4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH.sub.4.

The guanidine group of arginine residues may be modified by the formation of heterocyclic condensation products with reagents such as 2,3-butanedione, phenylglyoxal and glyoxal.

### IV. SCREENING ASSAYS

The role of GDE protein family members in mediating a GDE-related disorders makes the GDE protein family member an attractive target for agents that modulate these disorders to effectively treat, prevent, ameliorate, reduce or alleviate the disorders. Accordingly, the invention provides prescreening and screening methods aimed at identifying such agents. The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof or in animals.

In one embodiment, therefore, a prescreening method comprises contacting a test agent with a GDE protein family member. Such prescreening is generally most conveniently accomplished with a simple in vitro binding assay. Means of assaying for specific binding of a test agent to a polypeptide are well known to those of skill in the art. In one binding assay, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various screening formats are discussed in greater detail below.

Test agents including, for example, those identified in a prescreening assay of the invention can also be screened to determine whether the test agent affects the levels of GDE protein family members or RNA. Agents that reduce these levels can potentially reduce one or more GDE related disorders.

Accordingly, the invention provides a method of screening for an agent that modulates a GDE related disorder in which a test agent is contacted with a cell that expresses a GDE protein family member in the absence of test agent. Preferably, the method is carried out using an in vitro assay or in vivo. In such assays, the test agent can be contacted with a cell in culture or to a tissue. Alternatively, the test agent can be contacted with a cell lysate or fraction thereof (e.g., a membrane fraction for detection of GDE protein family members or polypeptides thereof). The level of (i) GDE protein family members; or RNA is determined in the presence and absence (or presence of a lower amount) of test agent to identify any test agents that alter the level. If the level assayed is altered, the test agent is selected as a potential modulator of a GDE related disorder. In particular embodiments, an agent that reduces or increases the level assayed is selected as a potential modulator of one or more GDE related disorders.

Cells useful in this screening method include those from any of the species described above in connection with the method of reducing a drug-related effect or behavior. Cells that naturally express a GDE protein family member are useful in these screening methods. Examples include PC12 cells, SH-SY5y cells, NG108-15 cells, IMR-32 cells, SK-N-SH cells, RINm5F cells, and MB cells. Alternatively, cells that have been engineered to express a GDE protein family member can be used in the method.

As noted above, screening assays are generally carried out in vitro, for example, in cultured cells, in a biological sample (e.g., brain, dorsal root ganglion neurons, and sympathetic ganglion neurons), or fractions thereof. For ease of description, cell cultures, biological samples, and fractions are referred to as “samples” below. The sample is generally derived from an animal (e.g., any of the research animals mentioned above), preferably a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

GDE protein family members can be detected and quantified by any of a number of methods well known to those of skill in the art. Examples of analytic biochemical methods suitable for detecting GDE protein family member, include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), receptor-linked immunosorbent assays (ELISAs), immunofluorescent assays, Western blotting, fluorescence resonance energy transfer (FRET) assays, yeast two-hybrid assays, whole or partial cell current recordings, and the like. Peptide modulators may be discovered or screened for example, by phage display. See U.S. Pat. Nos. 5,096,815; 5,198,346; 5,223,409; 5,260,203; 5,403,484; 5,534,621; and 5,571,698.

Methods for identifying lead compounds for a pharmacological agent useful in the treatment of a GDE related disorder comprise contacting a GDE protein with a test compound, and measuring GPI anchor cleavage activity. The GDE protein may also be a modified, e.g., a chimeric and/or a deletion mutant. The GDE protein may be isolated or may be in a membrane or an artificial membrane. The contacting may be directly or indirectly.

Methods of the invention also include methods for screening a therapeutic agent to treat, prevent, ameliorate, reduce or alleviate a GDE related disorder or symptoms thereof, comprising administering a test agent to a mouse having an over-expressed GDE protein.

Screening for GDE modulatory agents can be achieved by any one of several suitable methods including, but not limited to, contacting a cell comprising the GDE gene or functional equivalent or derivative thereof with an agent and screening for the modulation of GDE protein production or functional activity, modulation of the expression of a nucleic acid molecule encoding GDE or modulation of the activity or expression of a downstream GDE cellular target (e.g., GPI anchor cleavage). Detecting such modulation can be achieved utilizing techniques such as Western blotting, electrophoretic mobility shift assays and/or the readout of reporters of GDE activity such as luciferases, CAT and the like or observation of morphological changes.

The GDE gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate GDE activity, at either the nucleic acid or expression product levels, or the gene may require activation—thereby providing a model useful for, inter alia, screening for agents which up regulate GDE expression. Further, to the extent that a GDE nucleic acid molecule is transfected into a cell, that molecule may comprise the entire GDE gene or it may merely comprise a portion of the gene such as the portion which regulates expression of the GDE product. For example, the GDE promoter region may be transfected into the cell which is the subject of testing. In this regard, where only the promoter is utilized, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream GDE regulatory target, rather than GDE itself. Yet another example includes GDE binding sites ligated to a minimal reporter. For example, modulation of GDE activity can be detected by screening for the modulation of the functional activity in a cell. This is an example of an indirect system where modulation of GDE expression, per se, is not the subject of detection. Rather, modulation of the molecules which GDE regulates the expression of, are monitored.

These methods provide a mechanism for performing high throughput screening of putative modulatory agents such as the test agents comprising synthetic, combinatorial, chemical and natural libraries. These methods will also facilitate the detection of agents which bind either the GDE nucleic acid molecule or expression product itself or which modulate the expression of an upstream molecule, which upstream molecule subsequently modulates GDE expression or expression product activity. Accordingly, these methods provide a mechanism for detecting agents which either directly or indirectly modulate GDE expression and/or activity.

In one aspect, provided herein are methods for screening a therapeutic agent to treat, prevent, ameliorate, reduce or alleviate a GDE related disorder or symptoms thereof, comprising administering a test agent to a mouse having an over-expressed GDE protein, and measuring modulation of differentiation and/or GPI anchor cleavage activity. In one aspect, provided herein are methods for identifying lead compounds for a pharmacological agent useful in the treatment of a GDE related disorder comprising contacting a cell expressing a GDE protein with a test compound, and measuring GDE expression, modulation, or differentiation or modulation of GPI-anchor cleaving activity (e.g., glycerophosphodiesterase activity).

In one aspect, provided herein are methods for identifying lead compounds for a pharmacological agent useful in the treatment of a GDE related disorder comprising contacting a cell that does not express a functional amount of a GDE protein with a test compound, and measuring one or more of GDE expression, differentiation or GPI anchor cleavage.

In one embodiment, GDE expression or differentiation is measured by one or more of measuring protein or RNA expression, observing physical differentiation markers, measuring protein or RNA levels of one or more of NK-homeobox 6.1 (Gen Bank Accession No. NP_796374), Olig2 (Gen Bank Accession Nos.: AAH36245; BAB18907; NP_005797; Q9EQW6), homeobox factor 9 (Gen Bank Accession Nos.: NP_064328; NP_005506; P50219; Q9QZW9), p27 (Gen Bank Accession Nos.: BAA25263; NP_034005), Ngn2 (Gen Bank Accession Nos.: NP_033848; Q9H2A3; NP_076924; AAH36847), islet1 (Gen Bank Accession No.: NP_002193) or islet2 (Gen Bank Accession No.: NP_081673).

High throughput screening (HTS) typically uses automated assays to search through large numbers of compounds for a desired activity. Typically HTS assays are used to find new drugs by screening for chemicals that act on a particular receptor or molecule. For example, if a chemical inactivates a receptor it might prove to be effective in preventing a process in a cell which causes a disease. High throughput methods enable researchers to try out thousands of different chemicals against each target very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day. Examples include the yeast two-hybrid system and phage display. For examples of phage display see, U.S. Pat. Nos. 5,096,815; 5,198,346; 5,223,409; 5,260,203; 5,403,484; 5,534,621; and 5,571,698.

To achieve high-throughput screening, it is best to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known modulator (or activator) of a receptor for which modulators are sought, can be incubated with one sample of the assay, and the resulting decrease (or increase) in the receptor activity determined according to the methods herein. It will be appreciated that modulators can also be combined with the receptor activators or modulators to find modulators which inhibit the receptor activation or repression that is otherwise caused by the presence of the known the receptor modulator. Similarly, when ligands to a sphingolipid target are sought, known ligands of the target can be present in control/calibration assay wells.

Techniques for measuring the progression of binding reactions in multicontainer carriers are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., The Chemist's Companion:

A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol. I, pp. 155-194, CRC Press (1981).

In spectrofluorometric methods, receptors are exposed to substrates that change their intrinsic fluorescence when processed by the target receptor. Typically, the substrate is nonfluorescent and converted to a fluorophore through one or more reactions. As a non-limiting example, SMase activity can be detected using the Amplex® Red reagent (Molecular Probes, Eugene, Oreg.). In order to measure sphingomyelinase activity using Amplex Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (e.g., a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., Application of Fluorescence Polarization Assays in High-Throughput Screening, Genetic Engineering News, 17:27, 1997.

FP is particularly desirable since its readout is independent of the emission intensity (Checovich, W. J., et al., Nature 375:254-256, 1995; Dandliker, W. B., et al., Methods in Enzymology 74:3-28, 1981) and is thus insensitive to the presence of colored compounds that quench fluorescence emission. Fluoroecence Polarization (FP) and FRET (see below) are well-suited for identifying compounds that block interactions between sphingolipid receptors and their ligands. See, for example, Parker et al., Development of high throughput screening assays using fluorescence polarization: nuclear receptor-ligand-binding and kinase/phosphatase assays, J Biomol Screen 5:77-88, 2000.

Fluorophores derived from sphingolipids that may be used in FP assays are commercially available. For example, Molecular Probes (Eugene, Oreg.) currently sells sphingomyelin and one ceramide fluorophores. These are, respectively, N-(4,4-difluoro-5,7-dimethyl-4-bora-3a,4a-diaza-s-inda-cene-3-pentanoyl)s-phingosyl phosphocholine (BODIPY® FL C5-sphingomyelin); N-(4,4-difluoro-5,7-dimethyl-4-bora-3a,4a-diaza-s-inda-cene-3-dodecanoyl)-sphingosyl phosphocholine (BODIPY® FL C12-sphingomyelin); and N-(4,4-difluoro-5,7-dimethyl-4-bora-3a,4a-diaza-s-1-indacene-3-pentanoyl)-sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION fluorescence polarization system (Tecan A G, Hombrechtikon, Switzerland). General multiwell plate readers for other assays are available, such as the VERSAMAX reader and the SPECTRAMAX multi-well plate spectrophotometer (both from Molecular Devices).

Fluorescence resonance energy transfer (FRET) is another useful assay for detecting interaction and has been described previously. See, e.g., Heim et al., Curr. Biol. 6:178-182, 1-996; Mitra et al., Gene 173:13-17 1996; and Selvin et al., Meth. Enzymol. 246:300-345, 1995. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as an fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res. 38:2365-2373 (1997); Kahl et al., Anal. Biochem. 243:282-283 (1996); Undenfriend et al., Anal. Biochem. 161:494-500 (1987)). See also U.S. Pat. Nos. 4,626,513 and 4,568,649, and European Patent No. 0,154,734. One commercially available system uses FLASHPLATE scintillant-coated plates (NEN Life Science Products, Boston, Mass.).

The target molecule can be bound to the scintillator plates by a variety of well-known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells.

The assay can be measured immediately or allowed to reach equilibrium. Either way, when a radiolabel becomes sufficiently close to the scintillant coating, it produces a signal detectable by a device such as a TOPCOUNT NXT microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., Anal. Biochem. 257:112-119, 1998).

In one embodiment, GDE protein family members are detected/quantified using a ligand binding assay, such as, for example, a radioligand binding assay. Briefly, a sample from a tissue expressing GDE protein family members is incubated with a suitable ligand under conditions designed to provide a saturating concentration of ligand over the incubation period. After ligand treatment, the sample is assayed for radioligand binding. Any ligand that binds to GDE protein family members can be employed in the assay. Any of the GDE protein family member modulators discussed above can, for example, be labeled and used in this assay. An exemplary, preferred ligand for this purpose is ¹²⁵I-omega-conotoxin GVIA. Binding of this ligand to cells can be assayed as described, for example, in Solem et al. (1997) J. Pharmacol. Exp. Ther. 282:1487-95. Binding to membranes (e.g., brain membranes) can be assayed according to the method of Wagner et al. (1995) J. Neurosci. 8:3354-3359 (see also, the modifications of this method described in McMahon et al. (2000) Mol. Pharm. 57:53-58).

Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence GDE polypeptide(s) in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In certain embodiments, GDE polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads TM), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., 3H, 1251, 35S, 14C, or 32P), receptors (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, late; etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In preferred embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Changes in GDE protein family member subunit expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In one embodiment, amplification-based assays can be used to detect, and optionally quantify, a polynucleotide encoding a GDE protein of interest. In such amplification-based assays, the mRNA in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR(RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of the GDE mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of GDE mRNA can be full-length or less than the full-length of these polynucleotides. Shorter probes are generally empirically tested for specificity. Preferably, nucleic acid probes are at least about 15, and more preferably about 20 bases or longer, in length. See Sambrook et al. for methods of selecting nucleic acid probe sequences for use in nucleic acid hybridization. Visualization of the hybridized probes allows the qualitative determination of the presence or absence of the GDE mRNA of interest, and standard methods (such as, e.g., densitometry where the nucleic acid probe is radioactively labeled) can be used to quantify the level of the GDE polynucleotide.). A variety of additional nucleic acid hybridization formats are known to those skilled in the art. Standard formats include sandwich assays and competition or displacement assays. Sandwich assays are commercially useful hybridization assays for detecting or isolating polynucleotides.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). See also, for example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

Hybridization assays according to the invention can also be carried out using a MicroElectroMechanical System (MEMS), such as the Protiveris' multicantilever array.

GDE RNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

The invention also provides a screening method based on determining the effect, if any, of a test agent on the level of the depolarization-induced inward current mediated by GDE protein family members. Agents that reduce this current can potentially reduce one or more drug-related effects and/or behaviors. Conversely, agents that increase this current can potentially enhance such drug-related effects and/or behaviors.

The current can be measured using any available technique. An indirect measurement of current can be carried out described by McMahon et al. (2000) Mol. Pharm. 57:53-58). In this method, cells are loaded with a dye that fluoresces in the presence of (such as fura-2 AM) prior to depolarization. Cells are generally also preincubated in the presence or absence of an GDE protein family member-specific modulator (e.g., 1 uM omega-conotoxin GVIA) to determine the extent of the current that is attributable to GDE protein family members. Cells are subsequently depolarized by incubation in a 50 mM KCl buffer in the continued presence or absence of the modulator. The resulting current can then be calculated based on fluorescence, as described by Solem et al. (1997) J. Pharmacol. Exp. Ther. 282:1487-95. Ruiz-Velasco and Ikeda (J. Neuroscience (2000) 20:2183-91 describe the direct measurement of currents using a whole-cell variant of the patch-claim technique, which can also be employed in the present invention.

Cells useful for screening based on current include any of those described above in connection with screening based levels of GDE protein family members or polypeptides or RNA or described below in the Examples.

In one embodiment, the test agent is contacted with the cell in the presence of the drug. The drug is generally one that produces one or more undesirable effects or behaviors, such as, for example, sedative-hypnotic and analgesic drugs. In particular embodiments, the drug is ethanol, a cannabinioid, or an opioid.

In a preferred embodiment, generally involving the screening of a large number of test agents, the screening method includes the recordation of any test agent selected in any of the above-described prescreening or screening methods in a database of agents that may modulate a drug-related effect or behavior. The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

When a test agent is found to modulate one or more GDE protein family members, or RNA, a preferred screening method of the invention further includes combining the test agent with a carrier, preferably pharmaceutically acceptable carrier, such as are described above. Generally, the concentration of test agent is sufficient to alter the level of GDE protein family members or RNA, differentiation and/or GPI anchor cleavage activity. This concentration will vary, depending on the particular test agent and specific application for which the composition is intended. As one skilled in the art appreciates, the considerations affecting the formulation of a test agent with a carrier are generally the same as described above with respect to methods of reducing a drug-related effect or behavior.

Preferred compositions for use in the therapeutic methods of the invention inhibit the GDE protein family member function by about 5% based on, for example, compound state analysis techniques or modulatory profiles described infra, more preferably about 7.5% or 10% inhibition or initiation of differentiation and/or GPI anchor cleavage activity of the cell, and still more preferable, at least about 20%, 30%, 40%, 50%, 60%, 70%, 80%, 90% or 100% initiation or inhibition of differentiation and/or GPI anchor cleavage activity.

### V. METHODS OF USING GDE MODULATORS

The GDE modulators described herein have in vitro and in vivo diagnostic and therapeutic utilities. For example, these molecules can be administered to cells in culture, e.g., in vitro or in vivo, or in a subject, e.g., in vivo, to treat, prevent or diagnose a variety of GDE-mediated diseases, disorders or conditions.

In one embodiment, the modulators of the invention can be used to detect levels of GDE. This can be achieved, for example, by contacting a sample (such as an in vitro sample) and a control sample with the GDE modulator under conditions that allow for the formation of a complex between the modulator and GDE. Any complexes formed between the molecule and GDE are detected and compared in the sample and the control. For example, standard detection methods, well known in the art, such as ELISA and flow cytometric assays, can be performed using the compositions of the invention.

Accordingly, in one aspect, the invention further provides methods for detecting the presence of GDE (e.g., hGDE) in a sample, or measuring the amount of GDE, comprising contacting the sample, and a control sample, with a GDE modulator (e.g., an antibody) of the invention, under conditions that allow for formation of a complex between the antibody or portion thereof and GDE. The formation of a complex is then detected, wherein a difference in complex formation between the sample compared to the control sample is indicative of the presence of GDE in the sample.

Also within the scope of the invention are kits comprising the compositions of the invention and instructions for use. In one embodiment, the kit comprises an anti-GDE antibody. The kit can further contain a least one additional reagent, or one or more additional antibodies (e.g., an antibody having a complementary activity which binds to an epitope on the target antigen distinct from the first antibody). Kits typically include a label indicating the intended use of the contents of the kit. The term label includes any writing, or recorded material supplied on or with the kit, or which otherwise accompanies the kit.

### VI. PHARMACEUTICAL COMPOSITIONS AND ADMINISTRATION

Accordingly, a pharmaceutical composition of the present invention may comprise an effective amount of a GDE modulator (e.g., a GDE2 or GDE3modulator). As used herein, the term “effective,” means adequate to accomplish a desired, expected, or intended result. More particularly, an “effective amount” or a “therapeutically effective amount” is used interchangeably and refers to an amount of a GDE modulator, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from a GDE-mediated disease, disorder or condition. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which a GDE modulator is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a GDE modulator together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising a GDE modulator may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising a GDE modulator, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0004 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. The second therapeutic agent can be a chemotherapeutic, in the case of cancers. A combination therapy regimen may be additive, or it may produce synergistic results (e.g., in cancer greater than expected for the combined use of the two agents).

The compositions can be administered simultaneously or sequentially by the same or different routes of administration. The determination of the identity and amount of the pharmaceutical compositions for use in the methods of the present invention can be readily made by ordinarily skilled medical practitioners using standard techniques known in the art. In specific embodiments, a GDE modulator of the present invention can be administered in combination with an effective amount of another therapeutic agent, depending on the disease or condition being treated.

In various embodiments, the GDE modulator of the present invention in combination with an another therapeutic agent may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the GDE modulator of the present invention in combination with another therapeutic agent are cyclically administered. Cycling therapy involves the administration of a first therapy (e.g., the GDE modulator) for a period of time, followed by the administration of a second therapy (e.g., another therapeutic agent) for a period of time, optionally, followed by the administration of perhaps a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### MATERIALS AND METHODS

Regulated Expression of GDE2.

The GDE2.APML mutant was constructed by mutating the residues APML located within the GDPD domain to GAHD (3). In ovo electroporations were carried out as described (1, 2). The chick β-actin promoter vector (pCAGGS) was used in all electroporations unless otherwise specified. Sparse expression of GDE2, RECK-CD2, full-length and secreted forms of RECK in chick spinal cords was induced using a binary Cre-lox based expression system where coding sequences were preceded by loxP-STOPloxP sites and coelectroporated with CMV-Cre expression plasmids at HH St12 (3, 22). In these experiments a hyperactive form of GDE2 was used to gain efficient motor neuron induction (GDE2C25S; 2). Embryos were labeled with BrdU for 30 minutes after 24 hours to delineate the lateral extent of the VZ, dissected, embedded and sectioned. A similar system was used for sequential RECK and GDE2 expression in HEK293T cells; however, CreER was used to obtain 4-hydroxy tamoxifen (4-OHT) inducibility of Cre activity and RECK coding sequences were flanked by two loxP sites and inserted upstream of the GDE2 coding sequence. Thus, RECK is expressed in the absence of 4-OHT, but 4-OHT incubation led to RECK excision and concomitant GDE2 expression.

In Situ Hybridization and Immunofluorescent Staining.

In situ hybridization and immunostaining analyses were performed as previously described (1, 2). Antibodies used: guinea pig-Olig2 (1:20,000; Ben Novitch); mouse anti-Isl2 (1:100; DSHB); goat anti-β-Gal (1:3000; Arnel); rat anti-BrdU (1:100; Sigma) Rabbit anti-Lbx1 (1:100; Martyn Goulding); rabbit anti-Brn3a (1:100; Abcam); rabbit anti-Engrailed (1:2000; Abcam); rabbit anti-Chx10 (1:2000; Thomas Jessell); Goat anti-Gata2/3 (1:100; SantaCruz). Detection of BrdU was carried out as described (2). Chick Hes5-1 and Blbp (FABP7) in situ probe sequences were respectively derived from the 3′ region of the coding sequence and from the 3′UTR sequence based on NCBI reference sequence NM_001012695 (GI:60593015) and NM_205308 (GI:148762966). RECK in situ probes were derived from ORF NCBI sequence XM_418897 (GI: 118086330).

Cell Counts.

Counts of Isl2+ cells in the ventricular zone of chick embryos were obtained from 10-20 sections/embryo (n=6-8 embryos). Construction of Gde2−/− animals and neuronal counts were performed as described (3).

ShRNA.

Short hairpin (sh) RNAs were cloned into the pSilencer1.0-U6 vector (Ambion). ShRNA target sequences were as follows: 5′ AATGGAATAAGCTGAGAGATT 3′ for RECK shRNA1 (SEQ ID NO:1); 5′AACCAGAAATGTGGAAGGCAA 3′ for RECK shRNA2 (SEQ ID NO:2); 5′ AATTCGCGCCTAGGTCCGAAC 3′ for control shRNA1 (SEQ ID NO:3).

Cell Culture and Western Blots.

HEK293T cells were cultured in DMEM with 10% FBS on polyethyleneimine (PEI)-coated 12 well plates and transfected using Fugene6 agent (Roche). 24 hours after transfection, medium was changed to serum free DMEM and cells were incubated for an additional 24 hours. Medium and cell lysates were collected separately, and secretion and expression of proteins were analyzed by western blot and quantified using ImageJ (NIH). Antibodies used in Western analyses were rabbit-anti Jag1 (1:1000, Santa Cruz); rabbit anti-CRD (1:250, gift from Paul Englund) and mouse anti-myc (1.5 μg/ml for IP, DSHB) and as described.

Triton X-114 Partitioning.

Triton X-114 (Sigma) partitioning of GPI-anchored proteins was performed as previously described with modifications (16). 2% Triton X-114 was pre-conditioned in 100 mM Tris-HCl, pH 7.4, 150 mM NaCl buffer. Partitioning of secreted proteins in medium: Culture medium was incubated with TritonX-114 buffer (final 1%) and was maintained on ice for 10 min with occasional mixing. Two phases were separated by raising the temperature to 300 C for 5 min, followed by centrifugation (3000×g, 3 min, RT). The detergent-rich pellet was collected for membrane-bound proteins. Detergent-poor supernatant was subject to repeated separation steps and collected. Extraction and partitioning of total protein from cells: Cells were washed with PBS twice and were incubated with 1% Triton X-114 buffer for 10 min at 4° C. Resuspended cells were incubated on ice for 10 min with occasional mixing and were centrifuged at 16,000×g for 15 min at 4° C. Supernatants were separated as described in partitioning of medium.

Radiolabeling of Cells.

One day after transfection of HEK293T cells, cells were labeled overnight with 20 μCi/ml of [³H] ethanolamine (Moravek Biochmicals) or 50 μCi/ml of [³H] myo-inositol (PerkinElmer) in serum free DMEM. For inositol labeling, inositol-free DMEM was used (MP biomedicals). The medium was immunoprecipitated (IPed) with a myc antibody for 3 hours, followed by a 1 hour incubation with Protein G. IP complexes were washed 4 times with PBST (1% Triton X-100). Radioactivity incorporated in IPed samples was measured using a liquid-scintillation counter and normalized to the amount of RECK IP detected by Western blot.

In Vitro Glycerophophodiester Phosphodiesterase (GDPD) Assay.

HEK293T cells were transiently transfected with GDE1 or GDE2. Two days later, post nuclear membrane fractions were prepared (11) and used in enzyme assays. Preparation of substrates and implementation of the enzyme-coupled spectrophotometric assay were as previously described (12) with modifications. 15 μg of membrane proteins were incubated with 0.5 mM substrate in 100 mM Tris buffer (pH 7.5, 10 mM MgCl2 or 5 mM CaCl2) at 30° C. for 30 min. The reaction was stopped by adding perchloric acid (final 0.5 N). After neutralization with potassium carbonate solution, the amount of glycerol-3-phosphate (G3P) was measured by the absorbance change at 340 nm in hydrazine buffer (0.2 M hydrazine, 0.5 M Glycine, pH 9.5, 2.87 mM EDTA, 2.5 mM NAD, 0.1 mg/ml of G3P dehydrogenase). G3P level was normalized to GDE1 level after subtracting background level without enzyme.

Chemical Synthesis.

Cyclic G[1,2] phosphate synthesis was performed using the protocol described (28).

Surface Biotinylation Assay.

Surface biotinylation assay was performed as previously described (29). Briefly, thirty hours after transfection of HEK293T cells with RECK/GDE and CreER constructs, cells were cooled on ice, washed twice with ice cold PBS++(1×PBS, 1 mM CaCl2, 0.5 mM MgCl2) and then incubated with PBS++ containing 1 mg/ml Sulfo-NHS-SSBiotin (Pierce) for 30 min at 4° C. Unreacted biotin was quenched by PBS++ containing 100 mM Glycine. Cultures were harvested in RIPA buffer, sonicated, and centrifuged at 132,000 rpm for 20 min at 4° C. The supernatant was rotated with Streptavidin beads (Pierce) for 2 hr at 4° C. Precipitates were washed with RIPA buffer three times and eluted with SDS gel loading buffer.

### Example 1

### To Obtain Pharmacological Modulators of 6-TM GDE Activity

Currently, specific pharmacological modulators for GPI-AP cleavage have not been identified due to poor characterization of GPI-AP cleaving enzymes and the absence of an in vitro assay system. The robust GPI-AP cleaving activity of 6-TM GDEs has allowed the establishment of a luciferase-based, high throughput drug screening assay (signal to noise ratio=44.6; signal to background ratio=7.0; Z′ score=0.6: an excellent assay category). Measuring the GDE3-dependent release of GPI-anchored luciferase into the medium, the present inventors screened 3,360 compounds of the Johns Hopkins Drug library (JHDL), which contains FDA approved and phase II clinical trial drugs. The present inventors have selected common hits, which reduce or increase the release of luciferase activity into the medium more than 3 standard deviations of DMSO controls from duplicated experiments and identified 163 inhibitors (4.9%) and 138 activators (4.1%). Since the assay targets the whole GPI synthesis pathway as well as GPI cleaving activity, it is crucial to identify the site of action. Candidate drugs could modulate different steps of synthesis and cleavage of GPI-APs and will be categorized as follow: drugs activate or inhibit (i) the GPI biosynthesis machinery, (ii) glycosylation, (iii) surface expression of GPI-AP or GDE3, (iv) GPI cleaving activity of GDE, or (v) GPI cleaving-independent secretion of GPI-AP. Among 163 inhibitors, 23 drugs have been prepared from fresh powder stocks and their sites of action were tested by established western blot analyses and results are as follows: 10 drugs (no effect), 9 drugs (reducing surface expression and/or glycosylation of GPI-APs), and 4 drugs (inhibiting GPI-cleaving activity). Among 138 activators, 21 drugs have been prepared from fresh powder stocks: 15 drugs (no effect), 4 drugs (partially induces GPI-cleaving activity), and 2 drugs (significantly induces GPI-cleaving activity).

### Example 2

### Drug Screening Continued

Firefly luciferase (Luc) was attached to the amino (N)-terminus of CNTF receptor (CNTFR) construct after a signal peptide. Luc-CNTFR was expressed in HEK293T cells by transient transfection with or without GDE3. The release of Luc-CNTFR into the medium by GDE3 was monitored by western blot analysis and luciferase assay (Promega). For high throughput drug screening assay, HEK293T cells transfected with Luc-CNTFR and GDE3 were seeded in 96 well plates and treated with drugs for 24 hours (10 μM in 0.1% DMSO). Johns Hopkins Drug library (JHDL) contains more than 3,000 compounds, which includes FDA-approved drugs and drug candidates that have entered phase II clinical trials. In each 96 well plate, there are 16 vehicle controls (1×PBS, 2% DMSO) and 80 different drugs (200 uM stock; 20×). Ten μl of the medium and cell lysate, lysed with 200 μl of 1× Passive Lysis Buffer (PLB, Promega) was mixed with 50 μl of LARII and luciferase activity was measured by a microplate reader (BioTek).

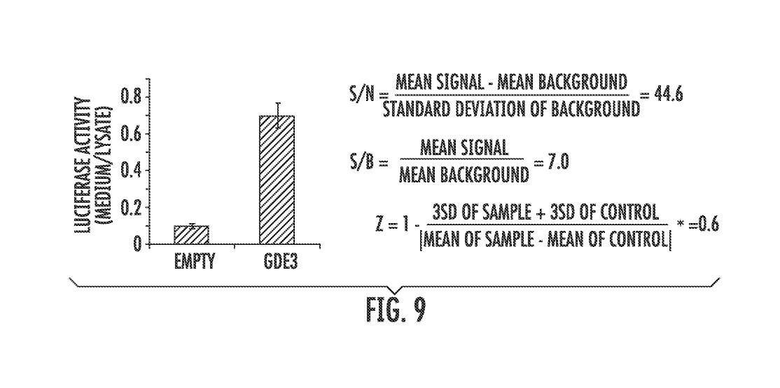

Luc-CNTFR was expressed in HEK293T cells with or without GDE3 and the luciferase activity in the medium was normalized to the lysate level to measure a release index (FIG. 9). GDE3 induces the release index by 7 fold, compared with empty control (transfected with Luc-CNTFR and empty vector). Signal to noise ratio, as measured by (mean signal of GDE3—mean empty)/standard deviation (SD) of empty control is 44.6. The assay is within an excellent assay category as Z′ score is measured 0.6 (Zhang et al., 4 J. MOL. SCREEN 67-73 (1999).

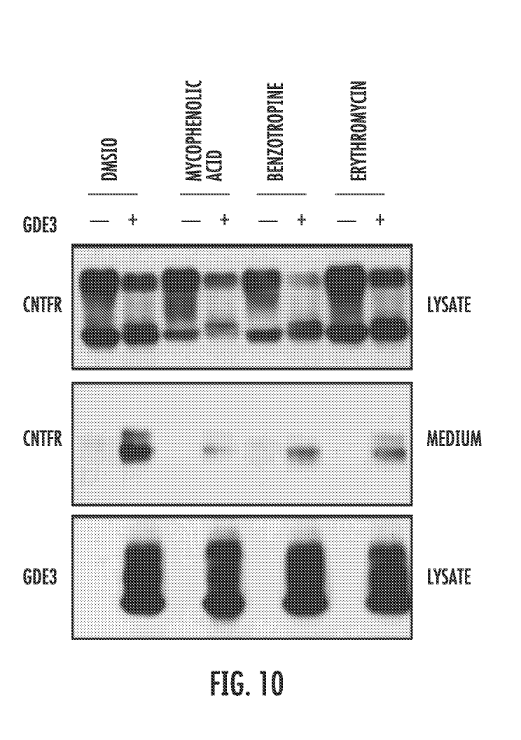

JHDL was used to screen drugs that affect the release of Luc-CNTFR into the medium. Drugs that increase or decrease the release index more than 3 SD of 16 DMSO controls of each plate were selected. Common hits of duplicated independent experiments were further analyzed. Among 3,360 drugs screened, we have identified 163 inhibitors and 138 activators. Among 163 inhibitors, 23 compounds were prepared from a fresh powder and their site of action was analyzed (10 μM in 0.1% DMSO, 24 hours treatment). To test whether drugs affect glycosylation and/or surface expression of CNTFR or GDE3, banding pattern of Western blot analysis and surface biotinylation assay were performed (FIG. 10). Out of 23 compounds tested, 9 drugs affect glycosylation pattern and reduced the surface expression of CNTFR. Four drugs (erythromycin, vitamin B12, thyroxine, nonivamide) were identified as inhibitor of the release of CNTFR without affecting expression and localization of CNTFR and GDE3. Among 138 activators, 21 compounds were tested from a fresh powder. Two drugs increase the release of CNTFR into the medium: fenbufen, pyrilamine.

### REFERENCES

1. M. Rao, S. Sockanathan, Transmembrane protein GDE2 induces motor neuron differentiation in vivo. Science 309, 2212-2215 (2005).
2. Y. Yan, P. Sabharwal, M. Rao, S. Sockanathan, The antioxidant Prdx1 controls motor neuron differentiation by thiol-redox dependent activation of GDE2. Cell 138, 1209-1221 (2009).
3. P. Sabharwal, C. Lee, S. Park, M. Rao, S. Sockanathan, GDE2 regulates subtype specific motor neuron generation through inhibition of Notch signaling. Neuron 71, 1058-1070 (2011).
4. N. Yanaka, Mammalian glycerophosphodiester phosphodiesterases. Biosci. Biotechnol. Biochem. 71, 1811-1818 (2007).
5. B. D'Souza, L. Meloty-Kapella, G. Weinmaster, Canonical and non-canonical Notch ligands. Curr. Top. Dev. Biol. 92, 73-129 (2010).
6. U. Marklund et al., Domain-specific control of neurogenesis achieved through patterned regulation of Notch ligand expression. Development 137, 437-445 (2010).
7. C. Y. Peng et al., Notch and MAML signaling drives Scl-dependent interneuron diversity in the spinal cord. Neuron 53, 813-827 (2007).
8. B. Zheng, C. P. Berrie, D. Corda, M. G. Farquhar, GDE1/MIR16 is a glycerophosphoinositol phosphodiesterase regulated by stimulation of G protein-coupled receptors. Proc Natl Acad Sci U.S.A. 100, 1745-1750 (2003).
9. T. Muraguchi et al., RECK modulates Notch signaling during cortical neurogenesis by regulating ADAM10 activity. Nat. Neurosci. 10, 838-845 (2007).
10. J. S. Rhee, L. M. Coussens, RECKing MMP function: implications for cancer development. Trends Cell Biol. 12, 209-211 (2002).
11. P. Lin et al., The mammalian calcium-binding protein, nucleobindin (CALNUC), is a Golgi resident protein. J. Cell Biol. 141, 1515-1527 (1998).
12. N. Ohshima et al., Escherichia coli cytosolic glycerophosphodiester phosphodiesterase (UgpQ) requires Mg2+, Co2+, or Mn2+ for its enzyme activity. J. Bacteriol. 190, 1219-1223 (2008).
13. L. Shi, J. F. Liu, X. M. An, D. C. Liang, Crystal structure of glycerophosphodiester phosphodiesterase (GDPD) from Thermoanaerobacter tengcongensis, a metal iondependent enzyme: insight into the catalytic mechanism. Proteins 72, 280-288 (2008).
14. D. Corda et al., The developmentally regulated osteoblast phosphodiesterase GDE3 is glycerophosphoinositol-specific and modulates cell growth. J. Biol. Chem. 284, 24848-24856 (2009).
15. M. J. Rebecchi, S. N. Pentyala, Structure, function, and control of phosphoinositidespecific phospholipase C. Physiol. Rev. 80, 1291-1335 (2000).
16. T. L. Doering, P. T. Englund, G. W. Hart, Detection of glycophospholipid anchors on proteins. Curr. Protoc. Protein Sci. Chap. 12, Unit 12.5. (2001).
17. J. D. Bangs, D. Hereld, J. L. Krakow, G. W. Hart, P. T. Englund, Rapid processing of the carboxyl terminus of a trypanosome variant surface glycoprotein. Proc. Proc. Natl. Acad. Sci. U.S.A. 82, 3207-3211 (1985).
18. S. E. Zamze, M. A. Ferguson, R. Collins, R. A. Dwek, T. W. Rademacher, Characterization of the cross-reacting determinant (CRD) of the glycosyl phosphatidylinositol membrane anchor of Trypanosoma brucei variant surface glycoprotein. Eur. J. Biochem. 176, 527-534 (1988).
19. M. G. Paulick, C. R. Bertozzi, The glycosylphosphatidylinositol anchor: a complex membrane-anchoring structure for proteins. Biochemistry 47, 6991-7000 (2008).
20. A. Gallet, L. Staccini-Lavenant, P. P. Thérond, Cellular trafficking of the glypican Dally-like is required for full-strength Hedgehog signaling and wingless transcytosis. Dev. Cell 14, 712-725 (2008).
21. D. Yan, Y. Wu, Y. Feng, S. C. Lin, X. Lin, The core protein of glypican Dally-like determines its biphasic activity in wingless morphogen signaling. Dev. Cell 17, 470-481 (2009).
22. B. Zhuang, Y. S. Su, S. Sockanathan, FARP1 promotes the dendritic growth of spinal motor neuron subtypes through transmembrane Semaphorin6A and PlexinA4 signaling. Neuron 61, 359-372 (2009).
23. A. J. Giráldez, R. R. Copley, S. M. Cohen, HSPG modification by the secreted enzyme Notum shapes the Wingless morphogen gradient. Dev. Cell 2, 667-676 (2002).
24. G. Kondoh et al. Angiotensin-converting enzyme is a GPI-anchored protein releasing factor crucial for fertilization. Nat. Med. 11, 160-166 (2005).
25. M. A. Davitz, D. Hereld, S. Shak, J. Krakow, P. T. Englund, V. Nussenzweig, A glycan-phosphatidylinositol-specific phospholipase D in human serum. Science 238, 81-84 (1987).
26. D. Yan, X. Lin, Shaping morphogen gradients by proteoglycans. Cold Spring Harb Perspect Biol. 1, a002493 (2009).
27. A. Fico, F. Maim, R. Dono, Fine-tuning of cell signaling by glypicans. Cell Mol. Life Sci. 68, 923-929 (2011).
28. L. Kugel, M. Halmann, Hydrolysis of glycero-1, 2-cyclic phosphate. J. American Chem. Soc. 89, 4125-4128 (1967).
29. J. H. Hu et al., Homeostatic scaling requires group I mGluR activation mediated by Homer1a. Neuron 68, 1128-1142 (2010).

1. A method of screening for antagonists of a glycerophosphodiester phosphodiesterase (GDE) protein comprising the steps of:
a. contacting a test agent with a cell that expresses the GDE protein; and
b. measuring the level of cleavage of glycosylphosphatidylinositol (GPI) anchors in the cell, wherein a test agent that decreases the measure cleavage level as compared to cleavage activity in a cell not contacted with the test agent identifies the test agent as an antagonist of GDE protein.

2. The method of claim 1, wherein the GDE protein is GDE2 or GDE3.

3. The method of claim 1, wherein the GDE protein is a GDE protein that cleaves GPI anchors.

4. The method of claim 1, wherein the agent is a small molecule, an antibody, polypeptide, a polynucleotide, an aptamer or an siRNA.

5. A method for treating a GDE-related disease, disorder or condition comprising the step of administering to a patient an effective amount of the antagonist of claim 1.

6. The method of claim 5, wherein the antagonist is erythromycin, vitamin B12, thyroxine, or nonivamide.

7. A method of screening for agonists of a GDE protein comprising the steps of:
a. contacting a test agent with a cell that expresses the GDE protein; and
b. measuring the level of cleavage of GPI anchors in the cell, wherein a test agent that increases the measure cleavage level as compared to cleavage activity in a cell not contacted with the test agent identifies the test agent as an agonist of GDE protein.

8. The method of claim 7, wherein the GDE protein is GDE2 or GDE3.

9. The method of claim 7, wherein the GDE protein is a GDE protein that cleaves GPI anchors.

10. The method of claim 7, wherein the agent is a small molecule, an antibody, polypeptide, a polynucleotide, an aptamer or an siRNA.

11. A method for treating a GDE-related disease, disorder or condition comprising the step of administering to a patient an effective amount of the agonist of claim 7.

12. The method of claim 11, wherein the agonist is fenbufen or pyrilamine.

13. A method for identifying a GDE protein modulator comprising the step of measuring GPI anchor cleavage activity of the GDE protein in the presence and absence of a test agent, wherein an agent that increases or decreases cleavage activity relative to cleavage activity in the absence of the test agent identifies the test agent as a GDE protein modulator.

14. The method of claim 13, wherein the GDE protein is GDE2 or GDE3.

15. The method of claim 13, wherein the test agent is a small molecule, an antibody, polypeptide, a polynucleotide, an aptamer or an siRNA.

16. A method for treating a GDE-related disease, disorder or condition comprising the step of administering to a patient an effective amount of the modulator of claim 13.

17. A method of screening for GDE modulators comprising the steps of:
a. contacting a cell that expresses GDE with a test agent;
b. assaying GPI anchor cleavage by GDE; and
c. comparing the assayed GDE activity to GDE activity in a cell that has not been contacted with the test agent, wherein a difference in the compared GDE activity identifies the test agent as a GDE modulator.

18. The method of claim 17, wherein the GDE protein is GDE2 or GDE3.

19. The method of claim 17, wherein the agent is a small molecule, an antibody, polypeptide, a polynucleotide, an aptamer or an siRNA.

20. A method for treating a GDE-related disease, disorder or condition comprising the step of administering to a patient an effective amount of the modulator of claim 17.

21. The method of claim 20, wherein the modulator is selected from the group consisting of erythromycin, vitamin B12, thyroxine, nonivamide, fenbufen, pyrilamine, and derivatives or biologically active fragments of the foregoing.

22. A method of screening for therapeutic agent useful in the treatment of GDE-mediated diseases comprising the steps of:
a. contacting a test agent with a GDE polypeptide; and
b. detecting the binding of the test agent to the GDE polypeptide.

23. The method of claim 22, further comprising:
c. contacting the test agent with a cell derived from a patient suffering from a GDE-mediated disease; and
d. determining the effect of the test agent on the cell.

24. The method of claim 23, wherein the agent is a small molecule, an antibody, polypeptide, a polynucleotide, an aptamer, or an siRNA.

25. A method for treating GDE-mediated disease comprising the step of administering to a patient an effective amount of the therapeutic agent of claim 23.

26. The method of claim 25, wherein the therapeutic agent is selected from the group consisting of erythromycin, vitamin B12, thyroxine, nonivamide, fenbufen, pyrilamine, and derivatives or biologically active fragments of the foregoing.

27. A GDE modulator that modulates the GPI anchor cleavage activity of GDE.

28. The method of claim 27, wherein the modulator is a small molecule, an antibody, polypeptide, a polynucleotide, an aptamer, or an siRNA.

29. The modulator of claim 27, wherein the modulator is selected from the group consisting of erythromycin, vitamin B12, thyroxine, nonivamide, fenbufen, pyrilamine, and derivatives or biologically active fragments of the foregoing.


SEQUENCE LISTING (3 sequences)

SEQ ID NO: 1 - DNA, 21 bp, Artificial Sequence
  RECK shRNA1
aatggaataagctgagagatt

SEQ ID NO: 2 - DNA, 21 bp, Artificial Sequence
  RECK shRNA2
aaccagaaatgtggaaggcaa

SEQ ID NO: 3 - DNA, 21 bp, Artificial Sequence
  Control shRNAI
aattcgcgcctaggtccgaac